(19) United States
(12) Patent Application Publication
(10) Pub. No.: US 2005/0208511 A1
(43) Pub. Date: Sep. 22, 2005

(54) DMBT1 as a clinical marker and uses thereof

(76) Inventors: George Allan, Bloomsbury, NJ (US); Sharon Hughes Tynan, Gaithersburg, MD (US); Jian-Zhong Guo, Three Bridges, NJ (US); Emmanuel Pacia, San Diego, CA (US); Scott Lundeen, Annandale, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/957,405

(22) Filed: Oct. 1, 2004

Related U.S. Application Data
(60) Provisional application No. 60/508,484, filed on Oct. 3, 2003.

(51) Int. Cl. C12Q 1/68; C07H 21/04; A61K 31/56

(52) U.S. Cl. 435/6; 536/24.3; 514/170

(57) ABSTRACT

The current invention provides methods for determining the estrogenic activity of a compound, which includes contacting an estrogen-responsive system having a gene under the control of a DMBT1 regulatory sequence with a test compound and determining how the test compound affects expression of the gene. The invention further provides for determining the progestogenic activity of a compound, which includes contacting an estrogen- and progesterone-responsive system having a gene under the control of a DMBT1 regulatory sequence with an estrogenic activity and a test compound and determining how the test compound effects expression of the gene. Nucleic acids and cell-based systems that include a portion of the DMBT1 regulatory sequence useful for these methods are also provided.

### FIELD OF THE INVENTION

This application claims priority from U.S. provisional application No. 60/508,484 entitled “DMBT1 as a clinical marker and uses thereof” filed Oct. 3, 2003 the contents of which are hereby incorporated by reference. The present invention relates to methods, compositions, and compounds useful for the identification and monitoring of compounds having an estrogenic or progestogenic effect. In the instant methods, regulatory and coding sequences of the gene deleted in malignant brain tumors 1 (DMBT1) are used for determining the estrogenic and progestogenic activities of compounds.

### BACKGROUND OF THE INVENTION

Hormone replacement therapy (HRT) represents an area of major importance in preventive medicine, and greatly affects personal well-being as well as public health. HRT has been used for a number of reasons, including the treatment of menopause, partial or full hysterectomy, and amenorrhoea. After menopause, estrogens are often given as a HRT to treat or prevent clinical indications such as moderate to severe vasomotor symptoms and vulvovaginal atrophy, osteoporosis, and is being tested in Alzheimer's disease and colon cancer. In some amenorrhoeic conditions, estrogen can be given to restore menses.

Although HRT using estrogen has many proven health benefits, it is also associated with some undesirable effects. These effects include an increased risk of uterine and mammary cancers, breast tenderness, and uterine bleeding. Co-adminstrative therapies have been used to reduce these side effects. To offset the increased risk of endometrial cancer, for example, progestins are often co-administered with estrogen during HRT. Unfortunately, other complications can arise using these cotherapeutic approaches. For example, progestin therapy is not currently considered appropriate for women without a uterus, and recent studies have demonstrated increased risks of cancers and strokes under the estrogen and progestin HRT regimen.

Newer advances in the area of HRT involve the use of compounds called Selective Estrogen Receptor Modulators (SERMs) as alternatives to estrogen in HRT. SERMs are drugs that can provide the beneficial effects of estrogen while avoiding other undesirable effects. For example, a SERM can provide a desirable estrogen agonist activity on bone tissue while providing an antagonist activity on uterine tissue. At present two SERMs are clinically available. The SERM tamoxifen is used for the prevention and treatment of breast cancer and the SERM raloxifene is used for the prevention of and treatment osteoporosis. These drugs, and other SERMs, typically bind to and affect the activity of the estrogen receptor.

Another class of compounds that can be useful in HRT regimens includes the Progesterone Receptor Modulators (PRMs), examples of which include mifepristone and onapristone. PRMs have been considered as adjuncts to estrogen replacement therapy and also for stand-alone treatment. PRMs are compounds that typically bind to the progesterone receptor and modulate the activity of the receptor, thereby affecting cellular activity. One of the important properties of PRMs is their anti-proliferative effects. For example, administering a PRM in the follicular phase of the menstrual cycle can inhibit endometrial proliferative activity. PRMs have a number of potential uses, including treatment of endometriosis and uterine fibroids, contraception, and hormone replacement therapy.

Despite advances in HRT research, there is a great need for novel SERMs and PRMs. The identification and use of novel SERMs and PRMs may provide therapy that is more suitable for particular conditions or for preventative treatment. These compounds can potentially provide the benefits of estrogen replacement therapy without the risks and side effects associated with current therapies. For example, novel SERMs may be able to promote activity in target tissues such as bone and neural tissues while leaving non-target tissue such as endometrial tissue and mammary tissue unaffected.

Additionally, there is a great need for new compositions and methods useful for the identification of novel SERMs and PRMs. These compositions and methods can also be valuable for clinically monitoring the activity of known or experimental SERMs and PRMs. Preferably, such methods will facilitate the rapid and accurate determination of compounds that are SERMs and PRMs.

Clearly, markers useful for the determination of estrogenic or progestogenic activity (as demonstrated by SERMs and PRMs, respectively) would represent a valuable contribution to the art. Such markers may advantageously lead to the development of new compounds for use in hormone replacement therapy.

### SUMMARY OF THE INVENTION

As described herein, the invention provides compounds, compositions and methods useful for identifying or screening compounds that have an estrogenic activity. One basis of the invention is the finding that a compound having estrogenic activity is able to upregulate the expression of the DMBT1 gene. Therefore, in one aspect, a method is provided that is useful for identifying a test compound that has an estrogenic activity. The method includes the steps of (a) contacting an estrogen-responsive system with a test compound; (b) obtaining information regarding the expression of a gene that is controlled by a DMBT 1-regulatory sequence from the estrogen-responsive system; and (c) using the information from step (b) to determine if the test compound has an estrogenic activity. In some cases, expression of the gene is measured relative to a control and an increase in the expression of the gene correlates with the test compound having an estrogenic activity.

The estrogen-responsive system, from which gene expression is measured, can be an animal, a portion of an animal, such as an estrogen-responsive tissue, or a cell. The gene controlled by the DMBT1 regulatory sequence can be DMBT1 itself or can be a heterologous gene operably linked to the DMBT1 regulatory sequence.

In another aspect, the invention provides a method for determining if a test compound has a selective estrogenic activity. In this method two different estrogen-responsive systems are contacted with the same test compound and gene expression, which is controlled by a DMBTL1-regulatory sequence, is measured from each system. The test compound has selective estrogenic activity if one of the estrogen-responsive systems shows an increase in gene expression and the other shows a decrease or no expression.

In another aspect, selective estrogenic activity can be determined by contacting two different estrogen-responsive systems and measuring gene expression controlled by a DMBT1-regulatory sequence in one system and measuring a different indicia of estrogenic activity (such as tissue growth) in the other system. The test compound has selective estrogenic activity if one of the estrogen-responsive systems shows, for example, no increase in gene expression and the other shows, for example, a positive response, such as tissue growth.

In yet another aspect, the invention provides a method for determining the progestogenic activity or anti-progestogenic activity of a compound. In this method an estrogen- and progesterone-responsive system is contacted with an estrogenic compound and also contacted with a test compound. Expression of a gene under the control of a DMBT1-regulatory sequence is measured from the system that has been contacted with the estrogenic and test compounds. The information obtained from the measurement of gene expression can be compared to a standard and used to determine if the test compound has a progestogenic activity or an anti-progestogenic activity.

In other aspects, the invention provides a way to monitor estrogenic and progestogenic activities in a subject. The method includes the steps of treating a subject, obtaining information regarding the expression of DMBT1 from the treated subject, and using the information on the expression to determine an estrogenic or a progestogenic activity.

In yet another aspect, the invention provides an estrogen-responsive system that includes a nucleic acid having a DMBT1 regulatory sequence operably linked to a reporter gene. The estrogen-responsive system can be a cell that includes an exogenous nucleic acid, such as a plasmid, having a DMBT1-regulatory operably linked to a reporter gene. The cell typically expresses a functional estrogen receptor, which can be expressed endogenously or exogenously. In a related aspect, the invention also provides an estrogen- and progesterone-responsive system that includes a nucleic acid having a DMBT1 regulatory sequence operably linked to a reporter gene. Cells of this system typically express both a functional estrogen receptor and a functional progesterone receptor.

In yet another aspect, the invention provides isolated heterologous nucleic acid sequences that include a portion of the DMBT1 regulatory sequence operably linked to a reporter gene. In particular, useful isolated heterologous nucleic acids have a DMBT1 regulatory sequence that include at least nucleotides 840-872 of SEQ ID NO. 1 operably linked to a reporter gene. These nucleic acids can allow for upregulation of the reporter gene in response to an estrogenic signal. Other useful isolated heterologous nucleic acids include a DMBT1 regulatory sequence that consists of all or a portion of nucleotides 1-2259 of SEQ ID NO:1, or a variant thereof, including nucleotides 840-872 of SEQ ID NO.1, operably linked to a reporter gene.

### BRIEF DESCRIPTION OF THE DRAWINGS

FIG. 1. Effects of estrone, SERMs, and an estrogen antagonist on uterine weight in rats (E: estrone, T: tamoxifen, R: raloxifene, and I: IC1182780).

FIG. 2. Effects of estrone, SERMs, and an estrogen antagonist on the expression of DMBT1 in the uterus of rats.

FIG. 3. Effects of estrone coadministered with SERMs, and an estrogen antagonist on the expression of DMBT1 in the uterus of rats.

FIG. 3B. Effects of increasing concentrations of estrogen or progestin (MPA) on the expression of DMBT1 in the uterus of rats.

FIG. 4. Effects of estrone and SERMs on uterine weight in rats (E: estrone; 5RA-DCC: 5R-Aryl-5,11dihydro-chromeno(4,3-c)chromene; and 5SA-DCC: 5S-Aryl-5,11dihydro-chromeno(4,3-c)chromene).

FIG. 5. Effects of estrone and SERMs on the expression of DMBT1 in the uterus of rats.

FIG. 6. Estrogenic induction of DMBT1/luciferase reporter constructs. Reporter constructs analyzed were: pDMBT-1-1/luc: DMBT1 promoter region from −1347 to +41 (upward pointing triangle); pDMBT1-2 (Downward pointing triangle)/luc: DMBT1 promoter region from −2921 to +41; oligo (solid diamond) (pDMBT1-3/luc): pDMBT1 promoter region from −2766 to −2734; pGL3 (open square): pGL3basic (control); pERE-luc (solid oval): chicken vitellogenin ERE sequence; and pTA (solid square); pTAbasic (control).

FIG. 7. Expression of DMBT1 mRNA in the endometrium of OVX monkeys treated with estrogen and various PRMs. (E2: estrogen; Mif: mifepristone; 17N11-DE: 17β-N-substituted-11β-dimethylaminophenyl-estra4,9-dien-3-one; 17β-N-substituted-11β-piperidinylphenyl-estra-4,9-dien-3-one: 17N11-PE). Results are provided as mean±standard deviation. Most test groups were n=3 monkeys. * refers to groups where n=1 and ** refers to test groups where n=2.

### DETAILED DESCRIPTION OF THE INVENTION

Unless defined otherwise, all technical and scientific terms used herein have the same meaning as commonly understood to one of ordinary skill in the art to which this invention belongs. Any methods, devices, and materials substantially similar or equivalent to those described herein can be used in the practice or testing of the invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

In this invention, certain terms are used frequently, which shall have the meanings as set forth as follows. These terms may also be explained in greater detail later in the specification.

As used herein, the terms “comprising”, “containing”, and “including” are used in their open, non-limiting sense.

“Estrogenic activity”, refers to the ability to produce an effect, or more than one effect, on a biological system that resembles an effect caused by estrogen. “Estrogen” refers to natural estrogens (including, but not limited to, estradiol, estrone, and estriol) and synthetic estrogens (including, but not limited to, ethinyl estradiol, diethylstilbesterol, and mestranol). Such an effect by estrogen is typically known as an “estrogen agonist activity”. A compound having estrogenic activity mimics at least one effect of estrogen and generally produces the effect by associating with the estrogen receptor to initiate the effect. It is understood that in a complex organism such as a mammal, estrogen has activity on different tissue types, and therefore, any one particular activity of estrogen on a tissue can be referred to as an estrogenic activity. Compounds referred to as SERMs typically have an estrogenic activity.

An “estrogenic effect” refers to a response within a cell, tissue, or organism that occurs when estrogen binds to its receptor. Examples of estrogenic effects include, independently, the translocation of estrogen receptors to nuclei; induced expression of estrogen-responsive genes, including DMBT 1; enhancement of nitric oxide production; cell proliferation, including, for example, proliferation of breast endothelial cells and endometrial cells; cell differentiation and growth, including, for example, enhanced growth and differentiation of neurites; tissue growth and growth of endometrial tissue; increased bone mineral density; changes in blood components including increases in high-density lipoprotein (HDL) cholesterol and triglycerides, decreases in low-density lipoprotein (LDL) cholesterol, and enhanced clotting; and increased inflammation.

“Estrogen-responsive system” is used in its broadest sense to refer to a collection of cells that initiate a change in gene expression or a change in protein phophorylation, for example, in response to estrogen. An “estrogen-responsive system” as used here in refers to single cells, collections of cells and therefore refers to tissues, organs or even complex multicellular organisms such as animals that are responsive to estrogen and where estrogen responsiveness can be tested.

A “selective estrogenic response” is demonstrated when a compound has different activities on two different estrogen-responsive systems or on two different portions of an estrogen-responsive system. Compounds that promote a selective estrogen response are commonly referred to as SERMs, some examples of which are provided herein. However, other compounds not commonly known as SERMs but which nonetheless can be shown to possess an estrogenic activity may also be able to promote a selective estrogenic response.

“Anti-estrogenic activity”, refers to the ability to produce an effect that is the opposite of that caused by estrogen and/or produce an effect that counters (reduces) an effect produced by estrogen. Compounds with “anti-estrogenic activity” include, for example, 1) compounds that reduce the expression of a gene or reduce the growth of a tissue, wherein estrogen normally increases production of the gene or increases growth of the tissue, 2) compounds that increase the expression of a gene or increase growth of a tissue wherein estrogen normally reduces production of the gene or reduces growth of a tissue; or 3) compounds that reducethe amount of an estrogenic response when the compound is administered concomitantly with estrogen. Many known compounds termed “estrogen antagonists” have “anti-estrogenic activity”.

“Progestogenic activity” refers to an effect, or more than one effect, on a biological system that resembles an effect caused by progesterone. As used herein, progesterone refers to progesterone and its synthetic analogs (progestins). Such an effect by progesterone is typically a “progesterone agonist activity”. A compound having progestogenic activity mimics at least one effect of progesterone and generally produces the effect by associating with the progesterone receptor to initiate the effect. It is understood that in a complex organism such as an animal, preferably a mammal, progesterone has activity on different tissue types, and therefore, any one particular activity of progesterone on a tissue can be referred to as progestogenic activity.

“Anti-progestogenic activity”, refers to the ability to produce an effect that is the opposite as that caused by progesterone and/or produce an effect that counters (reduces) an effect produced by progesterone. Generally, compounds having an anti-progestogenic activity can associate with the progesterone receptor and alter its activity. Some anti-progestogenic compounds or treatments can also reduce the effect of estrogen on a system. For example, some anti-progestogenic compounds can associate with the progesterone receptor and reduce the expression of a compound or reduce the growth of a tissue that is induced by estrogen. Many known compounds termed “progesterone antagonists” can block the effects of a compound having progestrogenic activity, and has anti-progestogenic activity

Compounds referred to as “progesterone receptor modulators” (PRMs) can bind to and alter the activity of the progesterone receptor. In some cases PRMs can modulate the effects of estrogenic and progestogenic compounds on a biological system. Some PRMs can have anti-progestogenic activity, such as progesterone antagonist activity.

“Progesterone-responsive system” is used in its broadest sense and refers to a collection of cells that initiate a change in gene expression or a change in protein phophorylation, for example, in response to progesterone. A “progesterone-responsive system” as used here in refers to single cells, collections of cells and therefore refers to tissues, organs or even complex multicellular organisms such as animals that are responsive to estrogen and where progesterone responsiveness can be tested.

“Estrogen- and progesterone-responsive system” is used in its broadest sense and refers to a collection of cells that initiate a change in gene expression or a change in protein phophorylation, for example, in response to both estrogen and progesteron. An “estrogen- and progesterone responsive system” as used here in refers to single cells, collections of cells and therefore refers to tissues, organs or even complex multicellular organisms such as animals that are responsive to estrogen and progesterone and where estrogen and progesterone responsiveness can be tested.

A “DMBT1 gene” refers to a gene that (1) encodes a protein having a sequence that has greater than about 60%, 65, 70, 75, 80, 85, 90, or 95% amino acid sequence identity, to human DMBT1 protein (Mollenhauer et al. (1997) Nat. Genet. 17: 32-39; GenBank protein accession No: NP_{—}015568.1); (2) encodes a protein capable of binding to antibodies, e.g., polyclonal or monoclonal antibodies, raised against a DMBT1 protein, such as the human DMBT1 protein described herein; (3) specifically hybridizes under stringent hybridization conditions to a nucleic acid molecule having a sequence that has greater than about 60%, 65, 70, 75, 80, 85, 90, or 95% nucleotide sequence identity, to human DMBT1 cDNA (GenBank nucleotide accession No: NM_{—}007329.1); or (4) can be amplified by primers that specifically hybridize under stringent hybridization conditions to a DMBT1 cDNA, such as the human DMBT1 cDNA described above. Stringent hybridization conditions are well known in the art (see for example, in Sambrook et al., Molecular Cloning: A Laboratory Manual, Second Edition, Cold Spring Harbor Laboratory, Cold Spring Harbor, N.Y., (1989)). A “DMBT1 gene” includes DMBT1 orthologs that have been identified in human (GenBank accession numbers NM_{—}017579 and AJ243212), rat (GenBank accession number U32681, 86% nucleotide identity to human), mouse (GenBank number U37438, 89% identity to human), cow (BF600097, 87% identity), and other animals. The gene has also been named glycoprotein (GP) 340, CRP-ductin, hensin and ebnerin.

A “DMBT1-regulatory sequence” refers to a nucleic acid sequence of the DMBT1 gene that can control the expression of the DMBT1 (or a DMBT1 ortholog) open reading frame in response to an estrogenic signal. In referring to aspects of the DMBT1 regulatory sequence, a 3715 nucleotide sequence of the 5′ region of the human DMBT1 gene is provided by SEQ ID NO: 1 (EMBL Accession No. AJ271916) and shown in Table 2. Nucleotide positions of the 5′ regulatory region of DMBT1 are herein referred to relative to the first nucleotide of the DMBT1 start codon (+1) and the position according to the numbering of nucleotides of SEQ ID NO: 1 in Table 2. Examples of DMBT1 regulatory sequences are discussed infra.

As used herein, a “variant” nucleic acid molecule of the invention is a nucleic acid molecule that has at least 80%, preferably at least about 90%, and more preferably at least about 95%, but less than 100%, contiguous nucleotide sequence homology or identity to the nucleotide sequence of the corresponding wild type nucleic acid molecule. Variant nucleic acids include nucleotide bases not present in the corresponding wild type nucleic acid molecule, such as 5′, 3′, or internal deletions or additions relative to the corresponding wild type polynucleic acid molecule. The variant polynucleic acids of the invention are DMBT1-regulatory sequences that are capable of promoting DMBT1 expression in response to an estrogenic signal.

“Heterologous” refers to a polynucleotide or polypeptide not natively found in or produced by the host cells.

It is understood that the terminology used herein is not intended to limit the scope of the present invention. It must be noted that as used herein and in the appended claims, the singular forms “a,” “an,” and “the” include plural reference unless the context clearly dictates otherwise. Thus, for example, a reference to “a cell” is a reference to one or more cells and includes equivalents thereof known to those skilled in the art and so forth.

In one general aspect, this invention provides methods, compositions, and compounds useful for the identification and/or monitoring of compounds and treatments that have or promote an estrogenic activity. The invention is at least in part based on the discovery that compounds having an estrogenic activity can upregulate DMBT1-regulatory sequence-controlled gene expression, such as the expression of the DMBT1 gene itself or of a reporter gene under the control of a DMBT1 regulatory sequence. In a related embodiment, the invention can be used to identify compounds that do not upregulate, or downregulate, DMBT1-regulatory sequence-controlled gene expression and therefore lack an estrogenic activity, or have an anti-estrogenic activity, respectively.

In another aspect, this invention provides methods, compositions, and compounds useful for the identification and/or monitoring of compounds and treatments that have or promote a progestogenic or anti-progestogenic activity. The invention demonstrates that compounds having a progestogenic or anti-progestogenic activity, when administered with an estrogenic compound, can reduce the estrogen-mediated upregulation of a DMBT1-regulatory sequence-controlled gene expression in certain estrogen- and progesterone-responsive systems. For example, a compound having a progestogenic activity can reduce the estrogen-mediated upregulation of a DMBT1-regulatory sequence-controlled gene expression in rat uterus; whereas a compound having an anti-progestogenic activity can reduce the estrogen-mediated upregulation of a DMBT1-regulatory sequence-controlled gene expression in monkey uterus.

The invention further provides estrogen-responsive systems that include a DMBT1-regulatory sequence which are useful for the identification and monitoring of compounds suspected of having estrogenic or anti-estrogenic activities. The invention also provides estrogen- and progesterone-responsive systems that include a DMBT1-regulatory sequence and that are useful for the identification and monitoring of compounds suspected of having anti-progestogenic or progestogenic activities.

Isolated nucleic acids that have a DMBT1-regulatory sequence operably linked to a reporter gene are also provided. The DMBT1 regulatory sequences are capable of upregulating expression of the reporter gene in response to an estrogenic signal.

DMBT1 is a complex protein structurally, and its function appears to be equally complex. Work to date has shown that it is involved both in mucosal protection and in epithelial differentiation. These functions have been defined in different species and in different tissues. For example, DMBt1 expression in the lung is associated with immune responses (Holmskov et al., Proc Natl Acad Sci USA 1999, 96:10794-10799; Madsen et al, 2003, Eur J Immunol. 33:2327-2336; Bikker et al, 2002, J Biol Chem 277:32109-32115; Prakobphol et al, 2000, J Biol Chem. 275:39860-39866) and in the liver, kidney and gut it is associated with cell differentiation (Bisgaard et al, 2002 Am J Pathol. 161:1187-1198; Vijayakumaret al., 1999, J Cell Biol. 144:1057-1067; Cheng et al, 1996, Anat Record 244:327-343). It is not clear if the two functions can always coexist in the same tissue. The present invention demonstrated that DMBT1 is expressed under the control of estrogen in uterine epithelium, a site with on-going cycles of proliferation and differentiation and has an important role in simulating immune activity in mucosal secretions.

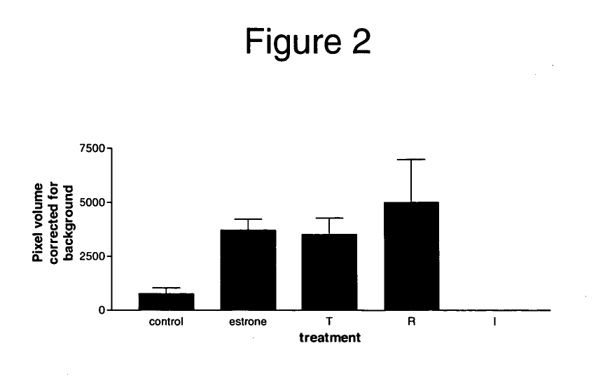

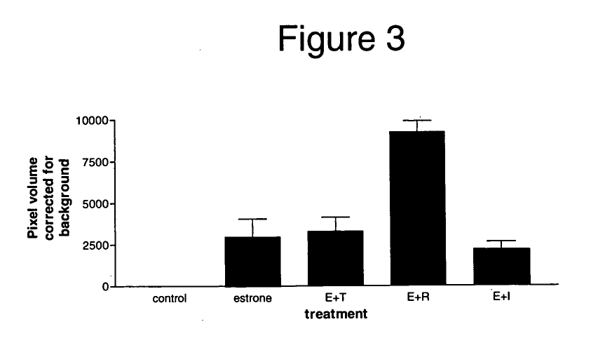

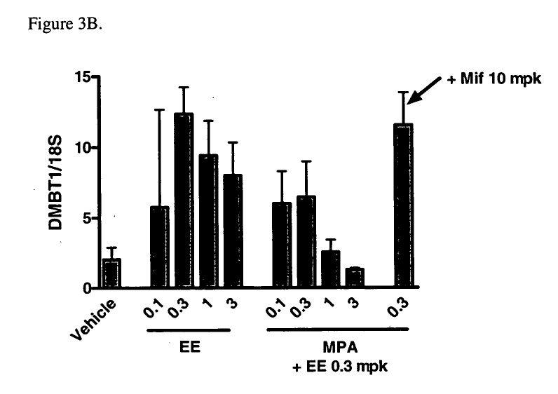

The present invention demonstrated that DMBT1 was strongly up-regulated by estrogen in monkey endometrium and in rat uterine epithelium (FIGS. 7 & FIG. 2). Gene induction of DMBT1 expression occurred rapidly in vivo, after one day of estrogen treatment in rats (FIG. 3B). The estrogenic induction of DMBT1 expression in monkey uterus was inhibited by mifepristone, a progestin antagonist (FIG. 7). The antiproliferative activity of mifepristone in estrogen-dominant monkey or rabbit uterus correlates with previous work (Wolf et al., 1989, Fertil Steril. 52:1055-1060; Slayden et al, 1993, Endocrinology 132:1845-1856; Chwalisz et al, 2000, Steroids 65:741-751). In addition, the estrogenic induction of DMBT1 expression in rat uterus was inhibited dose-dependently by a progestin. Further, the anti-estrogenic effect of the progestin was reversed by co-treatment of the rats with a progestin antagonist (FIG. 3B). These properties have been observed with other estrogen-dependent marker genes, such as that for complement component 3 (Lundeen et al., 2001, J Steroid Biochem Mol Biol. 78:137-143). The estrogenic induction of DMBT1 expression in rat uterus was also inhibited by co-treatment of the rats with ICI 182,780, an estrogen antagonist (FIG. 3A).

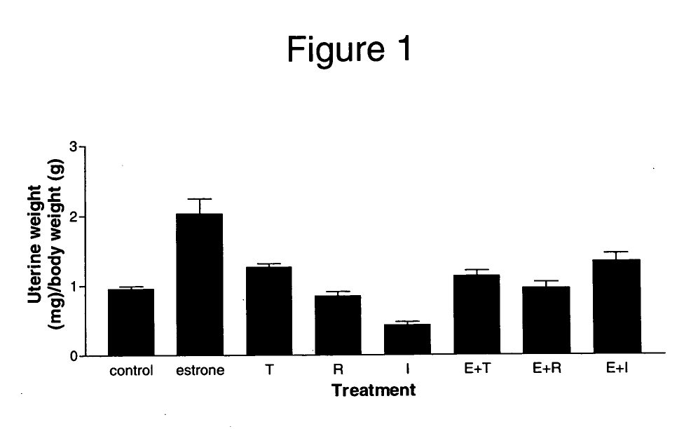

In addition, tamoxifen, a SERM that increases epithelial thickness in the uterus (Nephew et al, 2000, Proc Soc Exp Biol Med. 223:288-294) and is considered an estrogen agonist in that tissue, strongly stimulated DMBT1 expression in rat uterus (FIG. 2). Interestingly, the SERM raloxifene also stimulated DMBT1 expression in rat uterus (FIG. 2), particularly uterine epithelium (data not shown). Raloxifene is an estrogen antagonist in the rat uterus, and, in contrast to tamoxifen, does not have strong stimulatory effects by itself on uterine epithelium (Buelke-Sam et al., 1998, Reprod Toxicol. 12:217-221; FIG. 1).

Using immunohistochemistry, the present invention demonstrated that DMBT1 expression was limited to the epithelial cells of rat uteri (data not shown). This correlates with the findings of others, where DMBT1 was restricted to the epithelia of various tissues (Holmskov et al, supra; Mollenhauer, 2000, Cancer Res. 60:1704-1710; Bisgaard et al, supra; Vijayakumar et al., supra; Cheng et al supra; Mollenhauer et al., 2002, Cancer Det Prev. 26:266-274).

The present invention demonstrated the usefulness of DMBT1 for identification of compounds having estrogenic, anti-estrogenic, progestogenic, or anti-progestrogenic activities.

Ace and Okulicz (Ace et al., 1998, J Clin Endocrinol Metab. 83:3569-3573) reported that in intact monkeys, DMBT1 was up-regulated during the progesterone-dominant phase of the menstrual cycle in intact monkeys, with little or no detectable expression in the endometrium during the estrogen-dominant phase. There, they further showed that DMBT1 gene expression, which was analyzed by in situ hybridization, was restricted to the endometrial stroma. Recently, the same group published a new paper indicating that monkey DMBT1 expression decreased during the progesterone-dominant phase (Ace and Okulicz, 2004, Reprod Biol Endocrinol. 2:54 http://www.rbej.com/coritent/2/1/54).

In one embodiment, the invention provides a method for identifying and/or screening for compounds that have at least one estrogenic activity. The method includes the steps of (a) contacting an estrogen-responsive system with a test compound; (b) detecting gene expression from a gene operably linked to a DMBT1-regulatory sequence from the estrogen-responsive system as a measure of estrogenic activity.

In certain embodiments, step (b) can include measuring expression of the gene relative to a control and additionally identifying test compounds resulting in altered levels of gene expression relative to methods performed without the test compound.

In another specific embodiment, information regarding the expression of a gene controlled by a DMBT1-regulatory sequence can be obtained to determine if a test compound does not demonstrate an estrogenic activity or demonstrate an anti-estrogenic activity. In order to determine this, step (b) can further include correlating no detectable increase or a reduction in gene expression in response to the test compound with no detectable estrogenic activity or an anti-estrogenic activity, respectively. This method can be particularly useful for identifying compounds that have selective estrogenic activity (SERMs). For example, compounds that have been previously shown to demonstrate an estrogenic activity (for example, the stimulation of osteoid tissue) can be tested to determine if expression of a gene controlled by a DMBT1-regulatory sequence is not changed or, alternatively, downregulated in response to the test compound in a different estrogen-responsive system (such as endometrial tissue or mammary tissue, or cells derived from these tissues). Using these methods it is possible to identify SERMs having estrogenic activity on particular tissues. Other methods for the identification of compounds having selective estrogenic activity are provided below.

As indicated above, the estrogen-responsive system utilized in the present method can be a cell, a collection of cells including tissues, complex multicellular organisms that are responsive to estrogen. Typically, the estrogen-responsive system includes cells having estrogen receptors making the cells responsive to estrogen, as defined herein. When a ligand of an estrogen receptor (for example, an estrogenic compound) binds to the estrogen receptor a cellular signal can be initiated. This signal can affect the DMBT1 regulatory sequence causing upregulation of a gene linked to the DMBT1 regulatory sequence. Thus, the estrogen responsive system includes cells that contain genes operably linked to DMBT1 regulatory sequences. Methods to detect expression of the linked gene is measured as a function of the particular gene. Where DMBT1 is the gene, estrogen effects can be monitored. Where the genes are reporter genes, the cells preferably comprise recombinant constructs having reporter genes operably linked to DMBT1 regulatory sequences. Alternatively the cells are cells endogenously expressing DMBT1 under the control of an endogenous DMBT1 regulatory sequence. Exemplary reporter genes and methods to detect alterations in DMBT1 endogenous gene expression are discussed below. In the estrogen-responsive system, a test compound that binds to the estrogen receptor can upregulate, downregulate, or not affect expression of the gene that is controlled by the DMBT1-regulatory sequence.

An “estrogen receptor” as defined herein refers to any protein that binds to estrogen and the binding of which is capable of increasing or decreasing the activity of the estrogen signaling pathways. Preferably, an estrogen receptor is a member of the nuclear receptor gene family that binds estrogen, and that (1) has an amino acid sequence which shares greater than about 60%, 65, 70, 75, 80, 85, 90, or 95% amino acid sequence identity, to human estrogen receptor 1 (alpha) (Greene et al. (1986) Science 231:1150-4; GenBank protein accession No: NP_{—}000116), or human estrogen receptor 2 (beta) (Mosselman et al. (1996) FEBS Lett 392:49-53; GenBank protein accession No: NP_{—}001428); or (2) is capable of binding to antibodies, e.g., polyclonal or monoclonal antibodies, raised against an estrogen receptor, such as the human estrogen receptor 1 or 2 described herein.

As used herein, an estrogen receptor includes that of human and non-human mammals as described below. Human estrogen receptors, as referred to herein, include the alpha and beta isoforms as described herein, and any additional isoforms as recognized by those of skill in the art. A “functional estrogen receptor” as defined herein is a type of estrogen receptor that is capable of regulating gene expression transcriptionally.

In one specific embodiment the estrogen-responsive system is an animal. Animals include natural and genetically modified (transgenic) animals. The animal can be a human animal, such as a human being or a veterinary animal. Veterinary animals, include, but are not limited to, non-human animals of any kind such as a domestic animals, for example dogs and cats; farm animals, for example, cows, sheep, and pigs; laboratory animals, for example, mice, rats, monkeys, rabbits, and guinea pigs; and aquatic animals, for example, fish and turtles.

Transgenic animals can also be utilized in the methods of the invention. Previously created transgenic animals can be used or, alternatively, transgenic animals that are useful in the context of the current invention can be created. Useful transgenic animals can be created by introducing a transgene into an animal. Techniques for creating transgenic animals are known in the art and can be found in, for example, Pinkert, C. A. (Ed.) Transgenic Animal Technology: A Laboratory Handbook, 2nd edition, Academic Press (2003). Useful phenotypic alterations that can be provided by a transgene include alterations in estrogen or progesterone receptor expression, or alterations that provide variant estrogen and/or progesterone receptors. Other useful transgenes provide a DMBT1 regulatory sequence operably linked to a nucleic acid sequence of interest. A regulatory region is “operably linked” to a nucleic acid sequence if it is able to control the transcription of that sequence. Genes that can be operably linked to the DMBT1 regulatory region include, for example, detectable sequences and reporter genes, which are described herein.

Animals can also be surgically, pharmaceutically, or otherwise treated in order to produce a desired state in the animal prior to administration of the test compound. Such treatments can be used to reduce systemic levels of, or eliminate one or more naturally occurring substances in the body. For example, the animal can be treated to reduce systemic levels of, or eliminate hormones that have an effect on reproductive organs and glands. Hormones that have an effect on reproductive organs and glands include gonadotropins, for example follicle-stimulating hormone and luteinizing hormone, prolactin, estrogens, and etc. In other cases, treatments can be used to increase the presence of a naturally occurring substance in the body and can be useful when examining the effects of the test compound in the animal. Surgical treatments include, for example, the removal of organs or glands that produce hormones or other substances that can elicit autocrine or paracrine effects on tissues and cells in the animal. Examples of surgical procedures that can provide these results include ovariectomy, adrenalectomy and removal of testes. In some cases certain drugs can be administered to the animal prior to or during treatment of the test compound that reduce or eliminate one or more naturally occurring substances in the body.

Therefore, in preferred embodiments of the invention, the estrogen-responsive system is an animal that has been treated to reduce systemic levels of estrogen. A particularly useful treatment for reducing systemic levels of estrogen includes ovariectomy and the treatment can optionally include administering to the animal a compound that reduces synthesis of hormones that have an effect on reproductive organs and glands, such as nafarelin.

The methods described herein typically include a step of contacting the estrogen-responsive system, for example, an animal or an isolated group of cells or tissues with a test compound. The methods of this inention can be performed in vivo or in vitro. Where the contacting step comprises administering a test compound to an animal, the test compound can be administered in any suitable form and manner. The test compound can be prepared in a variety of forms, including a liquefied, gelled, lyophilized, dispersed, or solidified forms. It is anticipated that the preferred form will depend upon the physical characteristics of the test compound. The test compound can be administered orally, intravenously, intracerebrally, intramuscularly, intraperitoneally, intradermally, subcutaneously, intranasally, or intrapulmonary. The preferred mode of administration can depend upon the animal and the type of test compound administered and will be apparent to one of skill in the art. The test compound is typically administered to the animal at a dose and for a length of time sufficient for the test compound to exert its affect on the animal. This dose and duration of administration will also depend on the animal and the type of test compound administered.

The effects of the test compound can be measured in the animal in a variety of ways. It is understood that in a complex organism such as a mammal, estrogen has activity on different tissue types, and therefore, any one particular activity of estrogen on a tissue can be referred to as an estrogenic activity. Tissues that are responsive to estrogen include reproductive tissue such as uterine tissue, for example endometrial and myometrial tissues, mammary tissue, osteoid tissue, neural tissue, and vascular tissue, kidney tissue, liver tissue, lung tissue, smooth muscle, and skeletal muscle. Compounds that possess estrogenic activity can have an affect on one, some, or all of these tissues. In preferred embodiments, the estrogen-responsive system includes uterine tissue, or cells that are from a portion of the uterus.

An estrogenic effect can be determined by obtaining a sample of an estrogen-responsive tissue that has been contacted with the test compound and then measuring one or more features of the contacted sample. An estrogenic effect can also be determined by obtaining a sample that includes a component produced from an estrogen-responsive tissue contacted with the test compound. For example, a lipoprotein component present in a blood sample can be measured after a mammal has been administered the test compound. These types of samples from an organism can be referred to as “biological samples”.

A biological sample can include tissue, cells, and biological fluids isolated from a subject, as well as tissue, cells, and fluids present within a subject. The sample can be a “clinical sample” which is a sample derived from a patient. Such samples include, but are not limited to, sputum, blood, blood cells (e.g., white cells), amniotic fluid, plasma, semen, bone marrow, and tissue or fine needle biopsy samples, urine, peritoneal fluid, and pleural fluid, or cells therefrom. Biological samples can also include sections of tissues such as frozen sections taken for histological purposes. Exemplary biological samples include tissue biopsies taken from the endometria. A biological sample can also be referred to as a “patient sample.” A test biological sample is the biological sample that has been the object of analysis, monitoring, or observation. A control biological sample can be either a positive or a negative control for the test biological sample. Often, the control biological sample contains the same types of tissue, cells, and biological fluids as that of the test biological sample.

In another embodiment of the invention, information on the expression of a gene under the control of a DMBT1 regulatory sequence is measured from two or more different estrogen-responsive tissues, or cell types, in the animal. Advantageously, obtaining and analyzing this information can allow the determination of whether a test compound has a selective estrogenic activity (for example, whether the compound is a SERM). For example, a compound demonstrates selective estrogenic activity, if in one sample the test compound causes an increase in gene expression controlled by the DMBT1 regulatory sequence, and in the other tissue the test compound does not cause an increase or causes a decrease in gene expression controlled by the DMBT1 regulatory sequence.

Therefore, according to this embodiment, the invention provides a method to identify or screen for compounds that have selective estrogenic activity that includes the steps of: (a) contacting a first estrogen-responsive system with a test compound; (b) contacting a second estrogen-responsive system with the test compound; (c) obtaining information indicative of expression of a gene controlled by a DMBT1-regulatory sequence from the first estrogen-responsive system and obtaining information indicative of expression of a gene controlled by a DMBT1-regulatory sequence from the second estrogen-responsive system; and (d) correlating (i) an increase in expression in the first estrogen-responsive system and a decrease or no expression in the second estrogen-responsive tissue, or (ii) a decrease or no expression in the first estrogen-responsive system and an increase in expression in the second estrogen-responsive system, with a selective estrogenic activity. In one aspect of this embodiment the method includes contacting an animal with a test compound, detecting gene expression from a gene operably linked to a DMBT1 regulatory sequence in a first estrogen responsive tissue from the animal and detecting gene expression from a gene operably linked to a DMBT1 regulatory sequence in a second estrogen responsive tissue from the animal or from a second animal and correlating gene expression in the estrogen responsive systems with selective estrogenic activity.

In another embodiment of the invention, a selective estrogenic activity can be determined by measuring DMBT1 or DMBT1 regulatory sequence-controlled gene expression from one cell or tissue in response to a test compound and then measuring another estrogenic effect produced in or from a different cell or tissue. The other estrogenic effect can be any type of effect that is produced by an estrogenic compound and that is not DMBT1 or DMBT1 regulatory sequence-controlled gene expression. Types of effects include intracellular effects caused by the activation of the estrogen receptor, including the translocation of estrogen receptors to nuclei; induced expression of estrogen-responsive genes; enhancement of nitric oxide production; cell proliferation, including proliferation of breast endothelial cells and endometrial cells; cell differentiation and growth, including enhanced growth and differentiation of neurites; tissue growth, including growth of endometrial tissue; increase in bone mineral density; changes in blood components including increases in high-density lipoprotein (HDL) cholesterol and triglycerides, decreases in low-density lipoprotein (LDL) cholesterol, and enhanced clotting; and increased inflammation.

In this embodiment, DMBT1 or DMBT1 regulatory sequence-controlled gene expression and the other estrogenic effect are measured from different estrogen-responsive tissues or cell types (that is, from different estrogen-responsive systems). According to this embodiment, the different estrogen-responsive systems can be, for example, from within the same animal, from within different animals, from an animal and from a modified cell, from two different modified cell types, etc. Selective estrogenic activity can be shown where the test compound (a) increases DMBT1 or DMBT1 regulatory sequence-controlled gene expression from one estrogen-responsive system and has no estrogenic effect or has an anti-estrogenic effect on the other estrogen-responsive tissue, or (ii) decreases or does not affect DMBT1 or DMBT1 regulatory sequence-controlled gene expression from one estrogen-responsive system and has an estrogenic effect on the second estrogen-responsive system.

Therefore, according to this embodiment, the invention provides another method to identify or screen for compounds that have selective estrogenic activity that includes the steps of: (a) contacting a first estrogen-responsive system with a test compound; (b) contacting a second estrogen-responsive system with the test compound; (c) measuring expression of a gene controlled by a DMBT1-regulatory sequence in the first estrogen-responsive system; (d) measuring another estrogenic effect from the second estrogen-responsive system; and (e) correlating (i) an increase in expression from the first estrogen-responsive system and no estrogenic effect or an anti-estrogenic effect from the second estrogen-responsive tissue, or (ii) a decrease or no expression from the first estrogen-responsive system and the presence of an estrogenic effect from the second estrogen-responsive system with a selective estrogenic activity.

According to this embodiment, a step of contacting is generally performed before the step of measuring for the same estrogen-responsive system. Apart from this limitation the steps can be performed in any desired order. In some approaches the effect of the test compound on DMBT1-regulatory sequence-controlled gene expression is first determined; in other approaches the effect of the test compound to produce another estrogenic effect on a different estrogen-responsive system is first determined. It is understood that the order of the steps can depend on the type of compound to be identified or on other factors relevant to the method.

Measuring the estrogenic effect from the other estrogen-responsive system can be performed using any suitable technique. In one specific embodiment, the estrogenic effect can be measured directly from a sample of the other estrogen-responsive system, such as a cell or a tissue sample. For example, in response to the test compound a tissue sample can be measured for an increase in size or mass, or analyzed using biochemical, immunochemical, or microscopic techniques to assess, for example, cell proliferation in the tissue. In another specific embodiment, the estrogenic effect can be measured by determining the production of a component produced by the other estrogen-responsive system in response to the test compound. For example, an estrogen-responsive tissue such as osteoid tissue can produce components that appear in the blood or another bodily fluid of an animal in response to the test compound, such as secreted bone turnover proteins (for example, osteocalcin, alkaline phosphatase, and procollagen propeptides). These components can be analyzed to determine if the test compound has estrogenic activity. Changes caused by the estrogenic activity of a test compound can be compared against a suitable control to quantify the estrogenic activity.

As indicated above, a particularly useful embodiment includes measuring the ability of a test compound to cause cells to proliferate. Cell proliferation can be measured, for example, in mammary, osteoid, neural, or vascular tissue. As indicated, proliferation can be assessed by measuring the increase in the weight or size of a tissue relative to a control. Proliferation can also be determined microscopically by examining cell division or by using biochemical or immunological techniques which allow one to determine the presence of a proliferative marker, such as a cell surface protein or an intracellular molecule. Useful markers for cell proliferation include, but are not limited to, cell cycle proteins such as the cyclins and proliferating cell nuclear antigen (PCNA), signaling intermediates such as proliferation-associated gene product (PAG), nucleic acid binding proteins and transcriptional factors such as the mRNA binding protein HuR, and the like.

In some embodiments of the invention, expression of DMBT1 nucleic acid or DMBT1 protein is measured. The amount of DMBT1 mRNA in a biological sample can be measured using a number of techniques. For example, DMBT1 mRNA can be measured by contacting the biological sample with a compound or an agent capable of specifically detecting the DMBT1 mRNA. Useful techniques include contacting DMBT1 mRNA with a labeled nucleic acid probe capable of hybridizing specifically to the DMBT1 mRNA. For example, the nucleic acid probe specific for DMBT1 mRNA can be a full-length human DMBT1 cDNA as described herein, or a portion thereof, such as an oligonucleotide of at least 15, 30, 50, 100, 250 or 500 nucleotides in length of the human DMBT1, and sufficient to hybridize to a DMBT1 mRNA under stringent conditions. Preferably, a nucleic acid probe specific for DMBT1 mRNA will only hybridize to DMBT1 mRNA under stringent conditions, not to other nucleic acids present in the assayed biological sample.

The term “labeled”, with regard to the nucleic acid probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Other useful techniques for determining the amount of DMBT1 mRNA in a biological sample include performing a reverse transcriptase-polymerase chain reaction (RT-PCR). Complementary DNA (cDNA) can be prepared from a sample treated with the test compound and the DMBT1 cDNA amplified using oligonucleotide primers specific for the DMBT1 sequence and able to hybridize to the DMBT1 cDNA under stringent PCR conditions. Kits are commercially available that facilitate the detection of PCR products that incorporate detectable labels, for example SYBR™ Green PCR Core Reagents (Applied Biosystems, Foster City, Calif.).

Other useful techniques for determining the amount of DMBT1 mRNA in a sample include DNA microarray analysis and Northern hybridizations as described in, for example, Example 1 and Example 2, respectively.

The DMBT1 protein in a biological sample can be measured by contacting the biological sample with a compound or an agent capable of detecting the DMBT1 protein specifically. A preferred agent for-detecting a DMBT1 protein is an antibody capable of binding specifically to a portion of the polypeptide. In one preferred method, an antibody specific for DMBT1 coupled to a detectable label is used for the detection of DMBT1. Antibodies specific for DMBT1 can be polyclonal or monoclonal. A whole antibody molecule or a fragment thereof (e.g., Fab or F(ab′)₂) can be used.

Techniques for detection of a polypeptide such as the DMBT1 protein include enzyme linked immunosorbent assays (ELISAs), Western blots, immunoprecipitations and immunofluorescence. Details for performing these methods can be found in, for example, Sambrook et al (supra).

Other techniques for the in vivo detection of mRNAs or protein include fusion of DMBT1 regulatory sequences with a reporter gene described infra, in-situ hybridization and immunohistochemistry (to localized messenger RNA and protein in specific subcellular compartments and/or structures).

In addition, quantitative methods, such as positron emission tomography (PET) imaging, make possible the assessment by noninvasive means of the level of DMBT1 proteins in the living human organ (Sedvall et al., (1988) Psychopharmacol. Ser., 5:27-33). For example, trace amounts of the DMBT1 protein binding radiotracers are injected intravenously into the subject, and the distribution of radiolabeling in the uterus, breast, bone, vascular system, or brain of the subject can be imaged. Procedures for PET imaging as well as other imaging means are known to those skilled in the art (see review by Passchier et al., (2002) Methods 27:278).

In other embodiments, a reporter gene is placed under the control of the DMBT1 regulatory region and expression of the reporter gene is measured. As used herein, “a reporter gene” is a nucleic acid sequence other than the nucleic acid sequence of the DMBT1 coding region, and encodes a protein that can provide a detectable signal when expressed in the cell. It is contemplated that a wide variety of nucleic acid sequences can be used as a reporter gene. Useful nucleic acid sequences include naturally occurring sequences, recombinant sequences, and synthetic sequences that can be detected using, for example, nucleic acid probes, when the sequence is expressed in the cell. Reporter genes can provide detectable signals, such as emissions from fluorescent proteins. Other reporter genes encode proteins that are capable of catalyzing a specific reaction in the presence of a reagent, the product of the reaction providing a detectable signal. Other reporter genes encode unique proteins that are detectable using an affinity assay, for example, an inmunoaffinity assay.

In preferred embodiments a reporter gene is placed under the control of a DMBT1 regulatory sequence and this heterologous sequence is introduced into an estrogen-responsive cell. Reporter genes that can be used in the methods and compositions of the invention include, but are not limited to, genes encoding green fluorescent protein (GFP), β-galactosidase (lacZ), luciferase (luc), chloramphenicol acetyltransferase (cat), β-glucuronidase, neomycin phosphotransferase, and guanine xanthine phosphoribosyl-transferase.

In another embodiment of the invention, the estrogen-responsive system is a cell that expresses a functional estrogen receptor, and the cell expresses a functional estrogen receptor also includes, at least a portion of a DMBT1-regulatory sequence that can upregulate expression of an operably-linked gene when the estrogen receptor binds a estrogenic compound.

Therefore, according to the invention, compounds can be identified or screened for estrogenic activity by: (a) contacting a cell with a test compound, the cell having a (i) functional estrogen receptor and (ii) a nucleic acid having a gene under the control of a DMBT1 regulatory sequence; (b) obtaining information indicative of expression of the gene; and (c) using the information from step (b) to determine an estrogenic activity.

Accordingly, test compounds that produce an increase in expression of the gene under the control of a DMBT1 regulatory sequence can have an estrogenic activity; whereas test compounds that produce a decrease in gene expression in the presence of an estrogen can be correlated with an anti-estrogenic activity.

In this method, naturally occurring cells or modified cells can be used. Naturally occurring cells include cells that are isolated from estrogen-responsive tissue, such as uterine tissue, for example endometrial and myometrial tissues, mammary tissue, osteoid tissue, neural tissue, and vascular tissue. Isolation of these cells can be performed by biopsy or other techniques known in the art.

“Modified cells” refers to cells that have been manipulated to change a certain genetic or biochemical feature of the cell. For example, modified cells include transfected and transgenic cells having a heterologous nucleic acid. Therefore, in another embodiment, the invention also provides modified cells that are estrogen-responsive and have a heterologous nucleic acid that includes a DMBT1 regulatory sequence operably linked to a reporter gene sequence. Such cell types are particularly useful for screening compounds for estrogenic activity.

According to the invention, any cell type that can express a functional receptor and that can propagate an estrogenic signal driving expression of a gene from the DMBT1 regulatory sequence can be used. Cell types that endogenously express an estrogen receptor can be utilized and transfected with a heterologous nucleic acid having a DMBT1 regulatory sequence operably linked to, for example, a reporter gene. Eukaryotic cells are preferred cell types. In one preferred embodiment, the host cell is of uterine origin, such as an endometrial stromal cell like Ishikawa cells. In another preferred embodiment, the host cell is from breast, such as MCF-7 cells. In another preferred embodiment, the host cell is from bone, such as MG63 cells. In another prefeiTed embodiment, the host cell is from the vascular system, such as HUVEC cells.

In yet another preferred embodiment, the host cell is a brain cell, such as SK-N-MC cells. In addition, the use of simple eukaryotic cells such as Saccharomyces cerevisiae as the basis for an estrogen-responsive cell system is contemplated (see Jungbauer, A., and Beck, V. J., (2002) Chromatogr B Analyt Technol Biomed Life Sci, 777:167).

Cells can be transfected with a nucleic acid that is able to express an estrogen receptor, as described herein. The estrogen receptor can be expressed from a vector that is either stably or transiently transfected into the cell. Vectors suitable for estrogen receptor expression are known in the art and commercially available, from, for example, Promega (Madison, Wis.). In some cases it may be desirable to express a variant of an estrogen receptor in a cell. Certain estrogen receptor variants may express different phenotypes relevant to its activity, for example, constitutive activity, increased sensitivity, and decreased sensitivity. Some examples of estrogen receptor variants are shown by Chambraud et al. (1990) J. Biol. Chem. 265:20686-91.

Cells can also be transfected with a heterologous nucleic acid having a nucleic acid sequence of a reporter gene under the transcriptional control of a DMBT1 regulatory sequence. The nucleic acid sequence under the control of a DMBT1 regulatory sequence can be any type of sequence that is detectable using the methods as described herein. Useful nucleic acid sequence can encode proteins or enzymes as described herein that allow detection of the cell when the gene is expressed. Particularly useful reporter genes encode reporters such as luciferase (luc) are also described herein. In order to determine, for example, specificity and background, a second gene fusion comprising the same reporter gene but a different regulatory sequence (for example, a regulatory sequence for a gene that is not responsive to a estrogenic signal) can be used as a control to increase the specificity of the assay.

In one embodiment, a nucleic acid having at least a portion of the regulatory region of DMBT1 (SEQ ID NO:1; shown in Table 2) that is sufficient to drive expression of an operably linked gene in response to an estrogenic signal can be prepared and introduced into a cell.

According to the invention, a sequence of nucleotides from position −2766 to −2734 of the DMBT1 regulatory sequence (840-872 of SEQ ID NO:1) is able to drive expression of an operably linked gene in response to an estrogenic signal. Therefore, in one embodiment of the invention, a nucleic acid including 840-872 of SEQ ID NO:1, or a variant thereof, can be operably linked to a gene and introduced into an estrogen-responsive cell and used in a method to identify compounds that have estrogenic activity. A variant of this DMBT1-regulatory sequence can include nucleotide substitutions and/or deletions that do not substantially reduce the ability of the sequence to drive expression of the operably lined gene in response to an estrogenic signal. Variants of 840-872 of SEQ ID NO: 1 can be readily prepared using synthetic and recombinant techniques. For example, a variant of can include a nucleic acid sequence having about 60% nucleotide sequence identity, preferably about 65, 70, 75, 80, 85, 90, or 95% nucleotide sequence identity, to nucleotides 840-872 of SEQ ID NO: 1. Preferably the DMBT1 regulatory sequence of this invention will hybridize under high stringency conditions to SEQ ID NO:1 or having at least 85% sequence identify to any consecutive 30-mer within SEQ ID NO:1.

In addition, it also has been discovered that a portion of the DMBT1 regulatory sequence upstream of position −1347 (upstream of nucleotide 2259 of SEQ ID NO:1) provides an improved response to an estrogenic signal. Therefore, in a preferred embodiment of the invention, a nucleic acid including a sequence from nucleotide 1-2259 of SEQ ID NO: 1, a portion of 1-2259 of SEQ ID NO:1, or a variant thereof, including nucleotides 840-872 of SEQ ID NO:1, can be operably linked to a gene and introduced into an estrogen-responsive cell and used in a method to identify compounds that have estrogenic activity. More specifically, a DMBT1 regulatory sequence having a sequence upstream of position −2734 provides an improved response to an estrogenic signal. Therefore, in another preferred embodiment of the invention, a nucleic acid including a sequence from 1-872 of SEQ ID NO:1, a portion of 1-872 of SEQ ID NO. 1, or a variant thereof, including nucleotides 840-872 of SEQ ID NO: 1, can be operably linked to a gene and introduced into an estrogen-responsive cell and used in a method to identify compounds that have estrogenic activity.

In another embodiment, the invention provides an isolated nucleic acid sequence including a DMBT1 regulatory sequence operably linked to a reporter gene. The DMBT1 regulatory sequence is capable of upregulating expression of DMBT1 in response to an estrogenic compound. In one embodiment the DMBT1 regulatory sequence comprises nucleotides 840-872 of SEQ ID NO:1, or variants thereof (as described herein), operably linked to a reporter gene.

In a more preferred embodiment the nucleic acid includes a DMBT1 regulatory from nucleotide 1-2259 of SEQ ID NO: 1, a portion of 1-2259 of SEQ ID NO: 1, or a variant thereof, including nucleotides 840-872 of SEQ ID NO: 1, operably linked to a reporter gene. In another preferred embodiment the nucleic acid includes from nucleotide 1-2259 of SEQ ID NO: 1, a portion of 1-872 of SEQ ID NO. 1, or a variant thereof, including nucleotides 840-872 of SEQ ID NO. 1, operably linked to a reporter gene.

As stated herein, the invention also illustrates that compounds having a progestogenic activity or an anti-progestogenic activity, when administered with an estrogenic compound, can reduce the estrogen-mediated upregulation of a DMBT1-regulatory sequence-controlled gene. This aspect of the invention provides a basis enabling methods for the screening, monitoring, and/or identification of compounds that have progestogenic or anti-progestogenic activities.

Progesterone receptor modulators (PRMs) can include compounds that have progestogenic activity in some tissues and anti-progestogenic activity in others, and that can be identified using the methods described herein. One example of a therapeutically useful PRM is mifepristone, which can oppose the proliferative action of estrogen in the endometrium without concomitantly inducing a secretory state (as progestins do). PRMs have been envisioned for use in estrogen-containing hormone replacement therapy regimens, where they would counteract the stimulatory effect of estrogen, and as stand-alone treatments for endometriosis, whose symptomatology is estrogen-dependent.

In one embodiment, the methods described herein can allow for the identification of novel compounds having a progestogenic or anti-progestogenic activity, such as PRMs. PRMs can be identified by determining the expression of a gene controlled by a DMBT1 regulatory sequence. For example, a test compound which downregulates an estrogen-induced upregulation of a gene under the control of a DMBT1 regulatory sequence can have anti-progestogenic activity and be a PRM.

Therefore, in another embodiment, the invention provides a method for identifying or screening compounds that have progestogenic or anti-progestogenic activity. The method includes the steps of (a) contacting an estrogen- and progesterone-responsive system with an estrogenic compound; (b) contacting the estrogen- and progesterone-responsive system with a test compound; (c) obtaining information indicative of expression of a gene controlled by a DMBT1-regulatory sequence from the estrogen- and progesterone-responsive system; and (d) using the information obtained from step (c) to determine progestogenic or anti-progestogenic activity.

In one embodiment, step (c) can include measuring expression of the gene relative to a control, and step (d) can include correlating a decrease in the expression in response to the test compound with a progestogenic activity or an anti-progestogenic activity. Specifically, a test compound decreases the estrogen-mediated upregulation of a DMBT1-regulatory sequence-controlled gene expression can be either progestogenic or anti-progestogenic, depending on the estrogen- and progesterone-responsive system used. For example, a compound having a progestogenic activity can reduce the estrogen-mediated upregulation of a DMBT1-regulatory sequence-controlled gene expression in rat uterus; whereas a compound having an anti-progestogenic activity can reduce the estrogen-mediated upregulation of a DMBT1-regulatory sequence-controlled gene expression in monkey uterus.

It is further possible that in some estrogen- and progesterone-responsive systems, a progestin agonist and a progestin antagonist will not be distinguishable in terms of their effects on DMBT1 expression, i.e., both of them will reduce the estrogen-mediated upregulation of a DMBT1-regulatory sequence-controlled gene expression. Preferably, the method of the invention further comprises a step of measuring a progesterone-responsive effect that is different from the expression of a gene controlled by a DMBT1-regulatory sequence. For example, in primates, progestin agonists and antagonists have distinct effects on endometrial histology, i.e., the former induces a secretory phenotype by differentiation, while the latter induces a non-proliferative, non-secretory phenotype by preventing cell division. Therefore, in some embodiments, the method of the invention further comprises a step of measuring changes in endometrial histology or markers of endometrial secretion, or by measuring specific progestin-dependent gene markers in mammary gland. In rodents, a second progestogenic effect can be assayed by decidualization, a progesterone-dependent assay that measures the readiness of the uterus to accept an embryo.

The estrogen- and progesterone-responsive system can be a single cell, a tissue, or a complex multicellular organism that is responsive to both estrogen and progesterone. Typically, the estrogen- and progesterone-responsive system includes a cell that has both an estrogen receptor and a progesterone receptor. The estrogen- and progesterone-responsive system also includes a gene under the control of a DMBT1-regulatory sequence, wherein the expression of the gene is measurable. In the estrogen- and progesterone-responsive system, the DMBT1 regulatory sequence-controlled upregulation of gene expression by an estrogenic signal can be inhibited by an anti-progestogenic activity, such as the action of a progesterone antagonist on the progesterone receptor.

A “progesterone receptor” as defined herein refers to any protein that binds to progesterone and the binding of which is capable of increasing or decreasing the activity of progesterone signaling pathways. Preferably, the progesterone receptor is a member of the nuclear receptor gene family that binds progesterone and that (1) has an amino acid sequence which shares greater than about 60%, 65, 70, 75, 80, 85, 90, or 95% amino acid sequence identity, to human progesterone receptor form B (Kastner et al. (1990) EMBO J 9:1603-14; GenBank protein accession No: NP_{—}000917); (2) is capable of binding to antibodies, e.g., polyclonal or monoclonal antibodies, raised against a progesterone receptor, such as the human progesterone receptor form B described herein. As used herein, a progesterone receptor includes that of human and non-human mammals (e.g., animals of veterinary interest such as horses, cows, sheep, and pigs, household pets such as cats and dogs, as well as laboratory animals such as rats, mice, rabbits, guinea pigs and monkeys). Human progesterone receptors, as referred to herein, include the B isoform described herein, the A isoform which lacks the first 164 N-terminal amino acid residues of the B isoform, and any additional isoforms as recognized by those of skill in the art. A “functional progesterone receptor”, as defined herein is a progesterone receptor capable of regulating gene expression transcriptionally.

In one specific embodiment, the estrogen- and progesterone-responsive system is an animal, which includes natural and genetically modified (transgenic) animals as described herein. These animals can also be surgically or pharmaceutically treated in order to produce a desired state in the animal prior to administration of the test compound, also described herein.

The method includes a step of contacting the estrogen- and progesterone-responsive system with the test compound. This step can be performed before, at the same time, or after the step of contacting with the estrogenic compound. The timing of contacting or administration of the test compound can depend on a number of factors, including the characteristics of the test compound itself, the estrogenic compound, the doses of the test compound and estrogenic compound, etc. The estrogenic compound and the test compound can be administered in any suitable form and manner as described herein.

The effects of the test compound can be measured in the animal in a variety of ways. It is understood that in a complex organism such as a mammal, both estrogen and progesterone have activity on different tissue types. Therefore, according to this method, it is desirable to utilize an estrogen- and progesterone-responsive system that is a) responsive to an estrogenic compound and produces a measurable estrogenic effect in response to an estrogenic compound and also b) responsive to a progestogenic compound. In this way, an progestogenic or anti-progestogenic activity of the test compound can be assessed.

In another embodiment of the invention, DMBT1 expression, or expression of a gene under the control of a DMBT1 regulatory sequence, is measured in a tissue from the animal that has been contacted with the estrogenic compound and the test compound. In some cases it may be sufficient to determine DMBT1 or DMBT1 regulatory sequence-controlled gene expression from only one tissue, for example, from endometrial tissue, in order to determine if the test compound has a progestogenic or anti-progestogenic activity.

In another embodiment of the invention, DMBT1 or DMBT1 regulatory sequence-controlled gene expression is measured from two or more tissues in the animal. Typically, the two or more tissues are different and are estrogen- and progesterone-responsive. In this embodiment, measurement can allow the determination of whether a test compound has selective progestogenic activity (for example, whether the compound is a PRM/SPRM). In at least one tissue the test compound will demonstrate an anti-progestogenic effect, such as a decrease in DMBT1 expression in the presence of an estrogenic compound, and in the other tissue the test compound will demonstrate no effect or a progestogenic effect.

In another embodiment, selective progestogenic activity can be determined by measuring the DMBT1 or DMBT1 regulatory sequence-controlled gene expression from one tissue in addition to measuring another progestogenic effect of the test compound in another tissue, wherein both tissues are contacted with an estrogenic compound and the test compound. The other progestogenic effect can be any type of effect that is produced by a progestogenic compound and that is not DMBT1 or DMBT1 regulatory sequence-controlled gene expression. Types of effects include intracellular effects caused by the activation of the progesterone receptor, including the translocation of progesterone receptors to nuclei; induced expression of progesterone-responsive genes; stimulation and differentiation of epithelial endometrial cells in the uterus; luteal development, and maintenance of luteal structure-function.

DMBT1 gene expression can be measured from the estrogen- and progesterone-responsive tissue treated with estrogen and the test compound using any of the techniques as described herein. In addition, measurement of the expression of a gene controlled by a DMBT1 regulatory sequence can also be measured as described herein.

In another embodiment of the invention, the estrogen- and progesterone responsive system is a cell that expresses a functional estrogen receptor and a functional progesterone receptor. The cell that expresses these receptors also includes, at least, a portion of a DMBT1-regulatory sequence that can drive expression of an operably linked gene when the estrogen receptor binds an estrogenic compound. Therefore, according to the invention, compounds can be identified or screened for progestogenic or anti-progestogenic activity by (a) contacting a cell with an estrogenic compound, the cell having a (i) functional estrogen receptor, (ii) a functional progesterone receptor, and (iii) a nucleic acid having a gene under the control of a DMBT1 regulatory sequence; (b) contacting the cell with a test compound; (c) measuring expression of the gene relative to a control; and (d) correlating a decrease in the expression of the gene in response to the test compound with a progestogenic or an anti-progestogenic activity.

In this method, naturally occurring cells or modified cells can be used. Naturally occurring cells include cells that are isolated from estrogen- and progesterone-responsive tissue, such as uterine tissue, for example endometrial and myometrial tissues, mammary tissue and neural tissue.

In another embodiment, the invention also provides modified cells that are estrogen- and progesterone-responsive and have a nucleic acid that includes a DMBT1 regulatory sequence operably linked to a reporter sequence. Such cell types are particularly useful for screening compounds for anti-progestogenic activity.

According to the invention, any cell type that can express a functional receptor and that can propagate an estrogenic signal to the DMBT1 regulatory sequence can be used. Cell types that endogenously express an estrogen receptor and a progesterone receptor can be utilized and transfected with a nucleic acid having a DMBT1 regulatory sequence. Cell types that endogenously express an estrogen and progesterone receptors include T47D and ZR75-1 cells. Eukaryotic cells are preferred. In addition, the use of simple eukaryotic cells such as Saccharomyces cerevisiae, as described herein, is contemplated as the basis for an estrogen- and progesterone-responsive cell system. Cells can be tested for their ability to stimulate protein expression from a DMBT1 regulatory sequence by transfecting cells with a nucleic acid encoding a reporter operably linked to a DMBT1 regulatory sequence or by measuring estrogenic effects from the cells or tissues having an estrogen receptor.

In another embodiment, cells can be transfected with nucleic acids that are able to express an estrogen receptor, a progesterone receptor, or both. The estrogen receptor and progesterone receptor have been described herein as well as vectors that can promote their expression. Expression of variant estrogen receptors and variant progesterone receptors is also contemplated. Cells can also be transfected with a nucleic acid having a gene under the control of a DMBT1 regulatory sequence as described herein. Preferred aspects of the DMBT1 regulatory sequence are also described herein.

Subjects who are taking estrogen for hormone replacement therapy run an increased risk of developing uterine cancer due to unopposed stimulation of the endometrium. In one preferred embodiment, methods of the present invention can be used to monitor activities of an estrogen, SERM, or PRM in the uterus of the subject, wherein the biological sample described herein is from the uterus of the subjects, such as an endometrial biopsy sample. Test compounds suspected of having these activities can also be monitored. Methods to obtain an endometrial biopsy sample from a subject are known to those skilled in the art. For example, endometrial biopsy samples can be collected by transcervical cannulation from the cranial uterine body at defined stages of the estrus cycle using biopsy forceps. Endometrial biopsy samples can also be collected using methods such as the Tao Brush method (Wu et al. (2000) Am. J. Clin. Pathol., 114:412-418), the fractional curettage, or by open surgical incision.

Stimulation by estrogens or progestins can also cause an increased risk of developing breast cancer in the subjects. In another preferred embodiment, methods of the present invention can be used to monitor activity of an estrogen, SERM, or PRM in mammary glands of the subject, wherein the biological sample described herein is from the mammary glands of the subject. Test compounds suspected of having these activities can also be monitored. Methods to obtain a biological sample from the mammary glands of a subject are known to those skilled in the art. For example, intraductal aspiration or the conventional squeezing collection method can be used to collect discharges from the mammary gland. Other methods that can be used to collect a biological sample from the breasts of a subject include, but are not limited to, ex vivo fine-needle aspiration (FNA) (Eliasen et al. (1991), Mod. Pathol., 4:196-200), breast core needle biopsies (Ellis et al. (2002) Clin. Cancer Res., 8:1155-1166), and open surgical incision.

Optionally, methods of the present invention can be used to monitor activity of an estrogen, SERM, or PRM in bone tissue or the vascular system of the subject, preferably a laboratory animal such as a monkey, a rabbit, or a rat, wherein the biological sample described herein is from the bone or the vascular system of the subject. Test compounds suspected of having these activities can also be monitored. Methods are known to obtain a biological sample from the bone or the vascular system.

Besides affecting the hypothalamus and other brain areas related to reproduction, ovarian steroids have widespread effects throughout the brain, on serotonin pathways, catecholaminergic neurons, and the basal forebrain cholinergic system as well as hippocampal formation, a brain region involved in spatial and declarative memory (McEwen (2002) Recent Prog Horn Res. 57:357-84). Particularly, estrogen is thought to be protective in the aging brain, but little is known about what the mechanism of protection may be.

In yet another preferred embodiment, methods of the present invention can be used to monitor activity of an estrogen, SERM, or PRM in the brain of the subject, wherein the biological sample described herein is from the brain of the subject. Test compounds suspected of having these activities can also be monitored. Methods are known to those skilled in the art to obtain a biological sample from the brain of a subject. For example, using a biopsy needle, a biological sample from the brain can be obtained via imaging-guided biopsy, such as CT- or MR-guided biopsy. Also, brain tissue can be analyzed during autopsy.

### EXAMPLE 1

### Identification of Gene Expression Upregulated by Estrogen Treatment in the Monkey Endometrium

The following example demonstrates the identification of a particular gene having an expression pattern that was strongly upregulated, among a group of upregulated genes, in the endometrium of ovariectomized monkeys in response to estrogen treatment.

Briefly, tissue samples were taken from ovariectomized monkeys and from control animals, labeled cDNA was prepared from the tissues, and then the cDNA was hybridized to a microarray. The microarray results revealed a number of genes that were either upregulated or downregulated in comparison to the control sample. One gene that was strongly upregulated in the presence of estrogen was identified as the putative tumor suppressor gene DMBT1.

I. Animals And Drug Administration

All procedures involving animals were conducted in an animal facility fully accredited by the American Association for Assessment and Accreditation of Laboratory Animal Care (AAALAC) and in accordance with The Guide for the Care and Use of Laboratory Animals (NIH). Protocols were approved by the Eastern Virginia Medical School Internal Animal Care and Use Committee (IACUC).

Adult female cynomolgus monkeys (Macaca fascicularis) were obtained from Covance Research Products. Following one complete menstrual cycle and in the follicular phase of the subsequent menstrual cycle, the animals were ovariectomized by a mid-ventral laparotomy. Ovariectomized (OVX) animals were subject to estrogen treatment after the fourth week following ovariectomy.

To study the effect of estrogen on gene expression, groups of three OVX monkeys were given an estrogen or placebo implant (vehicle control) on Day One of a 19-day treatment cycle. Steady-state serum estradiol levels of 100 to 200 pg/mL were achieved by implantation.

Following this period, the OVX monkeys were euthanized and tissue biopsies were taken from endometria of the monkeys treated with estrogen and the placebo. (Details of the endometrial features in OVX monkeys and OVX+estrogen-treated monkeys is described in Example 4.) The biopsies were placed in sterile containers, snap frozen immediately in liquid nitrogen, and stored at −80° C. until processed. Total RNA was prepared from each endometrial tissue biopsy, as described below.

II. Preparation of Total RNA from Tissue Biopsies

Total RNA was prepared from endometrial tissue biopsies (approximately 50 mg each) using RNAzol™ (Tel-Test, Friendswood, Tex.), as follows. Two milliliters of RNAzol™ were used per 100 mg of tissue. Tissues were homogenized for 2 to 3 minutes using a Tissue Tearor homogenizer (Model No. 985-370; BioSpec Products, Bartlesville, Okla.), then transferred to a glass mortar bowl. The tissues were ground with at least 10 strokes of the pestle until a clear suspension was obtained. The homogenates were transferred to a polypropylene conical flask, to which 0.1 mL of chloroform per 1 mL of homogenate was added. The mixtures were then shaken vigorously for 1 minute. The suspensions were then transferred to a 2 mL microcentrifuge tube and centrifuged for 15. minutes at 12000 g and at 4° C. in a fixed angle rotor using a Biofuge 17R centrifuge (Baxter, Deerfield, Ill.). The aqueous phase was carefully transferred to a new microcentrifuge tube. An equal volume of isopropanol was added to the aqueous phase, mixed gently, and incubated at 4° C. for 15 minutes. RNA was pelleted by centrifuging the mixture for 15 minutes at 12000 g and at 4° C. as described above. The supernatant was removed and the pellet washed with 75% (v/v) ethanol (Pharmco Products, Brookfield, Conn.), followed by centrifugation at 7500 g for 8 minutes at 4° C. The supernatant was removed, and then the RNA pellet was air-dried briefly and resuspended in RNase-free water (Promega, Madison, Wis.). The samples were then treated with Amplification Grade RNase-free DNase I to remove any residual DNA (Invitrogen, Carlsbad, Calif.). Specifically, twenty micrograms of RNA were treated in a reaction volume of 0.1 mL, containing 1× DNase I reaction buffer and 10 units of DNase I. The reaction continued at room temperature for 15 minutes. After digestion, 0.35 mL of Buffer RLT from an RNeasy Mini Kit (QIAGEN, Valencia, Calif.) was added and mixed thoroughly, followed by 0.25 mL of 100% (v/v) ethanol. The mixture was loaded on an RNeasy Mini Kit column and spun at 10,000 rpm for 15 seconds in an ALC 4214 microcentrifuge. The flow-through was discarded and the RNA remaining in the column was washed twice with Buffer RPE, with centrifugation each time at 10,000 rpm for 15 seconds in an ALC 4214 microcentrifuge. The RNA was eluted into a collection tube with 30 μL RNase-free water. Isopropanol precipitation, ethanol washing, and centrifugation were performed as above. One hundred to two hundred micrograms of RNA were prepared from each tissue sample.

III. Probe Preparation and DNA Microarray Hybridization

An aliquot of each RNA preparation was used to generate probe to be hybridized to a first type of chip, which contained human complementary DNA sequences from 4475 separate gene identifiers. Redundancy in this gene set is about 25%; that is, about 3400 of the spotted DNAs represent unique genes (or clusters). Human and monkey coding sequences are over 95% conserved, on average; therefore, complications due to poor complementarity between monkey RNA and the human genes on the chip were not anticipated.

Chips were generated by depositing purified polymerase chain reaction-amplified DNA in 5 M sodium thiocyanate onto Corning GAPS slides (Corning, N.Y.) with a GenIII Array Sp\dot{o}tter (Molecular Dynamics, Sunnyvale, Calif.), followed by ultraviolet cross-linking at 500 mJ.

Labeled cDNA probe was then prepared from the endometrial RNA samples. RNA was heat denatured and labeled cDNA was prepared by reverse transcription in the presence of Cy3-deoxycytidine triphosphate (Amersham Pharmacia Biotech, Piscataway, N.J.). The RNA was removed by digestion with RNase A (Amersham Pharmacia Biotech), followed by purification using a QIAquick 96 PCR purification kit (QIAGEN). All probe preparations were normalized by relative fluorescence as measured on a Cytofluor (Applied Biosystems, San Diego, Calif.). The probe preparation was dried, then resuspended in Version 2 Hyb buffer (Amersham Pharmacia Biotech) containing 50% (v/v) formamide (JT Baker, Phillipsburg, N.J.) and 10% (v/v) Cot-1 DNA (Invitrogen). This mixture was heated to 95° C. for 2 minutes, cooled, and 50 μL were placed on two chips. The chips were covered with a coverglass and sealed with DPX (Fluka, Milwaukee, Wis.) and were incubated at 42° C. for 14 hours. A series of 5 minute washes followed, starting with 1×SSC containing 0.2% (w/v) sodium dodecyl sulfate at 42° C. and ending at 55° C. in 0.1×SSC containing 0.2% (w/v) sodium dodecyl sulfate. The chips were dried and scanned in a GenIII Array Scanner (Molecular Dynamics). Raw intensity data from the same experiment were normalized to the 75^{th} percentile across all chips. Each gene identifier was spotted twice on each chip and duplicate chips were hybridized to each labeled sample. Thus, quadruplicate data points were generated for each gene identifier (n=4).

IV. Analysis of Data from Microarray Hybridization

Normalized microarray spot intensities were analyzed using the OmniViz Pro™ software package (Version 1.611; OmniViz, Maynard, Mass.). Data were extracted from the DNA Chip 2.1 database (La Jolla Biolnformatics Database, RWJPRI, La Jolla, Calif.). Mean intensities, coefficients of variation and annotations were downloaded for the chip type used in the experiment. The intensity data threshold was set at 35 units; that is, values less than 35 units were increased to 35 units prior to ratio construction. Ratios were created by dividing the thresholded experimental values by the appropriate thresholded control value. Data were processed by k-means clustering (Quackenbush, Nat Rev Genet. (2001) 2:418-27) using a correlation metric, with the cluster number set to 65. Subsequently, data were filtered to remove any ratios equal to or below twofold (relative to one of the reference samples), and any gene identifiers with coefficients of variation across any of the four spot intensities of greater than 50%. Finally, filtered data were processed by average hierarchical clustering, using a correlation metric with the resulting dendrogram cut to yield 50 clusters.

V. The Putative Tumor Suppressor Gene DMBT1 Was Induced In Estrogen-Treated Monkey Endometrium

A total of 237 gene identifiers were up- or down-regulated greater than twofold in estrogen treated (Ovx/E₂) compared to vehicle treated (Ovx/Placebo) ovariectomized animals. This included genes that were previously known to be estrogen-regulated, such as PR (Touitou et al. (1989) Mol. Cell. Endocrinol., 66:231-238), with threefold up-regulation, and the insulin-like growth factor binding protein 3 (Liu et al., (1997), Mol. Hum. Reprod., 3:21-26), with tenfold down-regulation. Other genes are present that were not previously known to be estrogen-regulated. One gene of particular interest, not previously known to be estrogen regulated, and that was identified as being strongly induced by estrogen was the putative tumor suppressor gene DMBT1 (Deleted in Malignant Brain Tumors 1). The expression of DMBT1 was found to be up-regulated 37-fold by estrogen. Results of the gene expression data, including DMBT1 upregulation, were confirmed with a second round of hybridization experiments to a second type of chip using the same RNA samples. The second type of chip contains about 5600 unique gene identifiers, including all of those identifiers that are on the first type of chip.

According to these data, DMBT1 gene expression was strongly upregulated in response to estrogen in the endometrium and, therefore, an increase in DMBT1-regulated gene expression correlates with estrogen agonist activity.

To verify these results, quantitative RT-PCR was performed on some of the RNA samples. DMBT1 expression was found to be increased 3000- to 9000-fold following estrogen treatment. Mifepristone prevented the increase in DMBT1 mRNA levels in all three monkeys, as assessed by microarray analysis and PCR (Example 6). No DMBT1 expression was detected in gonadotropin-treated monkey endometria.

### EXAMPLE 2

### The Selective Estrogen Receptor Modulators Tamoxifen and Raloxifene Increase DMBT1 Expression in the Rat Endometrium

The following example demonstrates that DMBT1 expression was upregulated in rat endometrium in response to two compounds, tamoxifen and raloxifene, that have selective estrogen activity in vivo. The example also demonstrates that DMBT1 expression was not upregulated in rat endometrium in response to a compound having estrogen antagonist activity. Therefore, these data show that DMBT1-regulated gene expression can be used as a marker to differentiate a compound having estrogen agonist activity, and in particular a compound having selective estrogen activity, from a compound not having any estrogen activity, for example, an estrogen antagonist. Tamoxifen and raloxifene are SERMs that show estrogen agonist activity on bone while demonstrating weak estrogen agonist activity on the endometrium (Miller (2002) Curr. Pharm. Des,. 8:2089-2111). ICI 182780 is an estrogen antagonist that exerts its activity on a number of tissues having estrogen receptors.

I. Animal Models and Drug Treatment

Ten groups of 22-day-old female Sprague Dawley rats (three animals per group) were used to evaluate the effects of estrone, tamoxifen, raloxifene, and ICI 182780 on DMBT1 expression in the uterus. The following groups of rats were treated for three days as follows:

| Group | Treatment |
|---|---|
| (1) | None (control) |
| (2) | 70 μg/kg/day estrone |
| (3) | 1 mg/kg/day tamoxifen |
| (4) | 1 mg/kg/day raloxifene |
| (5) | 1 mg/kg/day ICI 182780 |
| (6) | 70 μg/kg/day estrone + 1 mg/kg/day tamoxifen |
| (7) | 70 μg/kg/day estrone + 1 mg/kg/day raloxifene |
| (8) | 70 μg/kg/day estrone + 1 mg/kg/day ICI 182780 |

All treatments were delivered orally in 0.5% Methocel. After treatment the animals were euthanized and the uteri were collected, weighed, frozen in liquid nitrogen, and stored at −20° C. RNA was purified from the frozen uteri using 1.0 mL of TRIzol Reagent (Invitrogen) and a PowerGen 25 tissue homogenizer (Fisher Scientific), according to the manufacturer's instructions.

Weights of the uteri were determined at necropsy and are shown in FIG. 1. In rats, uteri increase in size under the proliferative influence of estrogen, and this increase in size can be measured by weighing the tissues. As shown, treatment with estrone (E) doubles the uterine weight, while tamoxifen (T) treatment increases uterine weight only moderately, and treatment with raloxifene (R) did not significantly affect uterine weight either positively or negatively (weights of control and raloxifene-treated uteri were equivalent within the calculated margins of error). Treatment with the estrogen antagonist ICI 182780 (I) resulted in a significant decrease in uterine weight.

Co-treatment with estrone and tamoxifen (E+T) and estrone and raloxifene (E+R) resulted in lower uterine weights, as compared to estrone (E), and were similar to the uterine weights of tamoxifen (T)- and raloxifene (R)-treated uteri, respectively. Co-treatment with estrone and ICI 182780 (E+I) resulted in an average uterine weight that was approximately between that of estrone (E) and ICI 182780 (1) treatments alone. These results show that raloxifene and ICI 182780 are antagonists of estrogen-induced uterine weight increases, and that tamoxifen is a weak stimulator of uterine weight; ie, it is a weak estrogen agonist.

II. Analysis of Gene Expression

A. Sample Separation and Blotting

Northern analysis was performed to determine gene expression, including DMBT1 expression, in the uterus of control and treated rats. For gel electrophoresis, samples containing 2 μg of total RNA in a volume of 2 μL of water were added to 6 microliters of formaldehyde loading dye (Ambion) and incubated at 65° C. for 20 minutes. One microliter of a 0.1 mg/ml ethidium bromide solution was then added to each sample. Samples were loaded onto 1× MOPS, 1.25% SeaKem Gold Agarose Reliant gels (BioWhittaker Molecular Applications) and separated by electrophoresis in 1× NorthemMax 10× MOPS Gel Running Buffer (Ambion) at 70 volts until the dyes had migrated approximately one-third (xylene cyanol) and two-thirds (bromophenol blue), respectively, through the gel. The RNA was transferred to a Hybond-N membrane (Amersham) for seven days by capillary transfer blotting, using 10×SSC (0.15 M Sodium citrate, pH 7.0, 1.5 M NaCl), after which the membranes were stained with ethidium bromide, destained with water, and photographed for lane loading comparison. The membranes were then air-dried and kept at room temperature until hybridization.

B. Preparation of DMBTI Probe

A glycerol stock of a bacterial clone carrying a plasmid (pINCY) containing the rat ebnerin (DMBT1) gene was obtained from Incyte Genomics (ID#: 0000101155000011). The glycerol stock was streaked onto an LB agar+100 μg/mL ampicillin plate (KD Medical) and grown at 37° C. overnight. One colony was selected and used to inoculate 200 mL of Lennox L Broth (Sigma)+50 μg/mL ampicillin (Sigma). The liquid culture was grown overnight at 37° C. and the bacteria were collected by centrifugation. Purification of plasmid DNA was carried out using a QIAfilter plasmid maxi kit (QIAgen) according to the manufacturer's instructions.

Purified pINCY/DMBT1 was treated with the restriction enzymes EcoRI and NotI (Promega) for the preparation and isolation of a 1,174 bp DNA fragment containing the ebnerin probe (DMBT1). The products of the restriction enzyme reaction were separated by electrophoresis on a 1× TAE, 1% SeaKem Gold Agarose Reliant gel, and the band corresponding to the 1.174 kb DNA ebnerin clone was excised. The ebnerin DNA probe was purified from the gel slice using a QIAquick Gel Extraction Kit (QIAgen) according to the manufacturer's instructions. Bound DNA was eluted from the QIAgen column with 50 μL of elution buffer. The ebnerin DNA probe in the eluate was quantified by comparison to a DNA standard by gel electrophoresis. The ebnerin DNA probe was then diluted to a final concentration of 25 ng/μL.

Random-primed probe was made from 25 ng of the purified ebnerin DNA using a DECAprime II kit (Ambion) and Redivue α-³²P dATP (10 mCi/mL) (Amersham) according to the manufacturer's instructions, with the exception that the reaction was incubated for 30 minutes at 37° C. After adding EDTA to stop the reaction, the labeled probe was purified using a QIAquick Nucleotide Removal Kit (QIAgen) according to the manufacturer's instructions, with the final volume of eluted probe being 50 μL. Two microliters were counted in a scintillation counter to ensure optimal radionuclide incorporation (>2×10⁶ cpm/μL).

C. Probe Hybridization and Analysis

The blot membrane as described in section A was wet in 2×SSC buffer (Sigma) for 15 minutes, then pre-hybridized in 10 mL RapidHyb (Amersham) at 65° C. for 20 minutes. The probe was boiled for 10 minutes, and 25 μL was added to 10 mL of RapidHyb, pre-warmed to 65° C. The blot membrane was added to the diluted probe solution and incubated, with rotation, in a hybridization oven for approximately 10 hours.

The membrane was removed from the probe-containing solution and rinsed twice with 2×SSPE +0.5% SDS+0.5% pyrophosphate at room temperature, and then washed once, with gentle agitation, for 30 minutes at room temperature in the same solution. The membrane was then washed twice for 30 minutes each at 65° C. with gentle agitation in 0.5×SSPE+0.5% SDS+0.5% pyrophosphate. Following the last wash, the membrane was blotted on paper towels, wrapped in Saran Wrap and exposed. to a phosphor-imaging screen for 5 hours. The phosphor screen was scanned with a STORM 840 (Molecular Dynamics) using IQ Tools 2.2 software (Molecular Dynamics). The hybridized probe was visualized and quantified with Image Quant 5.2 (Molecular Dynamics).

The effects of the various treatments on DMBT1 expression are shown in FIGS. 2 and 3. FIG. 2 shows that DMBT1 expression was increased substantially in the estrone (E) treated uterine tissues (˜5-fold) as well as the tamoxifen (T)- and raloxifene (R)-treated uterine tissues (˜5-fold and ˜6.5-fold, respectively). However, treatment with ICI 182780 (I) resulted in undetectable levels of DMBT1-expression in the uterine tissue. FIG. 3A shows that DMBT1 expression increased slightly when tamoxifen was coadministered with estrone (E+T) and that the combination of estrone and raloxifene (E+R) resulted in an additive increase in DMBT1 expression. DMBT1 expression was moderately lowered when ICI 182780 was included with estrone treatment (E+T).

These results demonstrate that an increase in DMBT1 expression can be caused by compounds that have an estrogenic activity, such as estrone (E). Surprisingly, compounds that demonstrate selective estrogen activity, such as tamoxifen (T) and raloxifene (R) also strongly stimulate DMBT1 expression even though they had at best a modest effect on uterine growth. These results demonstrate that DMBT1 can be used to identify compounds that have an estrogenic activity, including compounds that have selective estrogenic activity such as SERMs, and in particular, compounds that have a positive effect on uterine growth.

This example also shows that differences in DMBT1 expression can allow differentiation of a compound having an estrogenic activity or a selective estrogenic activity from a compound having a negative estrogenic activity, for example, an antagonistic activity. Overall, the data indicate that DMBT1 expression or DMBT1 sequence-regulated expression can be correlated with estrogenic activity and selective estrogenic activity.

Furthermore, the data in this Example which illustrates the operability of the invention in a rat model system, along with the data from Example 1 detailing the operability of the invention in a monkey model system, provides support for the premise that an increase in DMBT1 expression or gene expression controlled by a DMBT1-regulatory sequence can be correlated with an estrogen agonist activity and selective estrogenic activity in mammals in general.

### EXAMPLE 3

### An Improved Selective Estrogen Receptor Modulator Increases DMBT1 Expression in the Rat Endometrium

The following example demonstrates that DMBT1 expression was upregulated in rat endometrium in response to a recently discovered compound, 5SA-DCC, that has selective estrogenic activity in vivo. The example also demonstrates that DMBT1 expression was not upregulated in rat endometrium in response to a compound having negative estrogenic activity and estrogen blocking activity in endometrial tissue but having estrogenic activity on blood and bone markers.

Therefore, similar to Example 2, these data show that DMBT1-regulated gene expression can be used as a marker to differentiate a compound having estrogenic activity, and in particular a compound having selective estrogenic activity, from a compound having negative estrogenic activity, particularly negative estrogenic activity on endometrial tissue.

I. Animal Models and Drug Treatment

Using a procedure similar to that described in Example 2, six groups of 22-day-old female Sprague Dawley rats (three animals per group) were used to evaluate the effects of estrone and the SERMs 5RA-DCC (5R-Aryl-5,11dihydro-chromeno(4,3-c)chromene) and 5SA-DCC: 5S-Aryl-5,11dihydro-chromeno(4,3-c)chromene) on DMBT1 expression. 5RA-DCC and 5SA-DCC are described in commonly assigned U.S. Patent Application No. 60/341957, entitled “Novel Heteroatom Containing Tetracyclic Derivatives as Selective Estrogen Receptor Modulators” by Kanojia, R. et al. The animals were treated for three days as follows:

| Group | Treatment |
|---|---|
| (1) | Control |
| (2) | 70 μg/kg/day estrone |
| (3) | 1.4 mg/kg/day 5RA-DCC |
| (4) | 1.4 mg/kg/day 5SA-DCC |
| (5) | 70 μg/kg/day estrone + 1.4 mg/kg/day 5RA-DCC |
| (6) | 70 μg/kg/day estrone + 1.4 mg/kg/day 5SA-DCC |

Administration of the treatments and collection of tissue samples were carried out according to the procedure described in Example 2. RNA was purified, blotted and hybridized to radioactively labeled rat ebnerin cDNA as in described in Example 2.

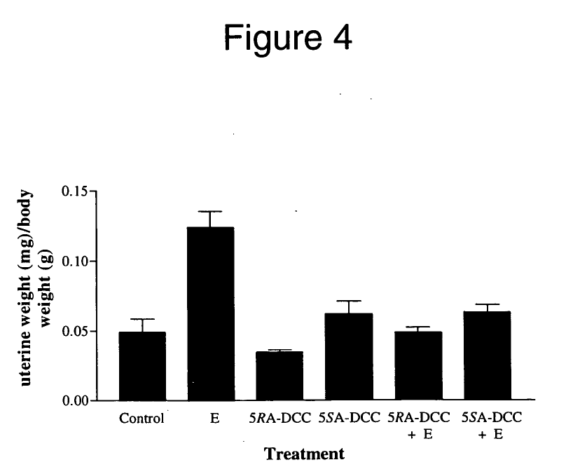

Effects of estrone, 5RA-DCC, and 5SA-DCC SERM treatment on rat uterine weights, corrected for body weight, are shown in FIG. 4. As shown in Example 2 (FIG. 1) and again in this example (FIG. 4), treatment with estrone (E) more than doubles uterine weight, reflecting the agonist activity of the hormone on this tissue. 5RA-DCC weakly stimulates uterine weight by itself, and antagonizes the uterine weight increase in the presence of estrone. In this regard, 5SA-DCC is similar to tamoxifen (FIG. 1).

In contrast to the other estrogenic compounds or selective estrogenic compounds that upregulate DMBT1 expression, 5RA-DCC demonstrates negative estrogenic activity and also acts as an estrogenic blocking compound with regard to the stimulation of uterine weight gain.

II. DMBT1 Expression

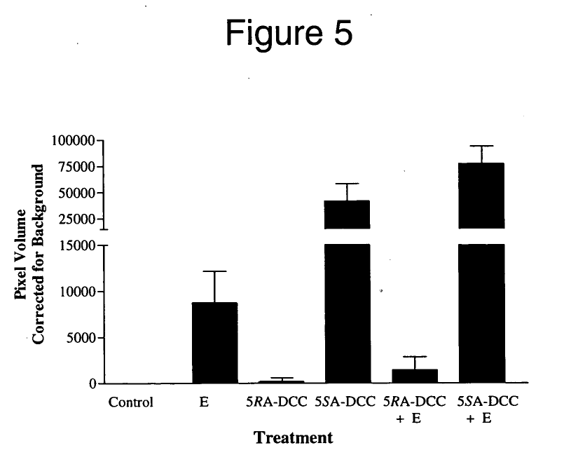

The effects of estrone, 5RA-DCC, and 5SA-DCC treatment on DMBT1 mRNA levels in rat endometrium are shown in FIG. 5. The effect of estrone treatment on DMBT1 mRNA expression correlates with that described in Example 2 (FIG. 2). 5SA-DCC strongly stimulated DMBT1 expression in the rat endometrium (more than 4-fold). Interestingly, the combination of 5SA-DCC and estrone provided a synergistic effect on DMBT1 expression, stimulating it more than 8-fold. These results are similar to the effects of tamoxifen, and more particularly raloxifene on DMBT1 expression.

In contrast, 5RA-DCC, by itself, does not stimulate DMBT1 gene expression. Interestingly, 5RA-DCC strongly blocks estrone-induced upregulation of DMBT1, decreasing the level of DMBT1 expression approximately 6-fold.

### EXAMPLE 4

### A DMBT1 Sequence-Regulated Cell-Based Assay Useful for Estrogen Agonist Identification

The following example demonstrates the construction of an estrogen-responsive cell carrying a nucleic acid having a DMBT1 regulatory sequence linked to a reporter sequence. In response to estrogen, the cell promoted transcription of the reporter sequence that was detectable using assay reagents. The example shows that this cell system is useful for detecting compounds that upregulate DMBT1 regulatory sequence-controlled transcription and correlate with estrogen agonist and selective estrogen agonist activity. The sequence of the 5′ region of the DMBT1 gene that includes DMBT1 regulatory sequences and sequences of oligonucleotide primers used for preparation of vectors that include DMBT1 regulatory sequences is shown in Table 2.

I. Construction of DMBT1 Regulatory/Luciferase Reporter Nucleic Acids

A. pDMBT1/1

A plasmid vector carrying a regulatory portion of the DMBT1 gene was constructed by cloning an upstream region of the human DMBT1 gene from −1347 to +41 (nucleotides 2259-3647 of SEQ ID No:1) into the pGL3basic plasmid (Promega). The resulting plasmid was named pDMBT-1-1/luc.

First, a 5′ portion of the DMBT1 gene from nucleotides −1347 to +41 was amplified by polymerase chain reaction (PCR) from human genomic DNA (Clontech) using 1× VENT DNA polymerase buffer, 40 pmoles of each primer, 40 nM dATP, 40 nM dGTP, 40 nM dCTP, 40 nM dTTP, 1 ng template DNA, and 4 units of VENT DNA polymerase (New England Biolabs) in a 100 μL reaction. The template was denatured at 94° C. for 5 minutes, followed by five sets of five cycles of touchdown PCR. The PCR parameters were: step 1, denaturation for 30 seconds at 94° C.; step 2, one minute annealing, the annealing temperature increasing from 60° C. to 70° C. in two degree increments every five cycles; step 3, extension at 72° C. for two minutes. After 30 cycles, there was a final five minute extension at 72° C. Oligonucleotides oDMBT1-1: 5′-GAATTCACGCGTGATCCCAGACCCAGCTGCATTATCATTCTC-3′ (SEQ ID No:2), and oDMBT1-2: 5′-GAATTCAAGCTTGGTGTGTCCTCTAGGGTGGTATATT TCTGC-3′ (SEQ ID No:3), were used as PCR primers. To facilitate molecular cloning, the restriction sites MluI and HindIII (underlined) were incorporated into SEQ IDs No: 2 and 3, respectively.

The 1.4 kb DMBT1 PCR product was purified from the PCR reaction mixture using a QIAquick Gel Extraction Kit (QIAGEN) according to the manufacturer's instructions. The purified DNA fragment was then ethanol precipitated and ligated to pGEM-T Easy (Promega) to create pDMBT1-1/GEM-T. The ligation reaction was transformed into JM109 competent cells (Promega) and plated on one LB agar+100 μg/mL ampicillin plate (KD Medical) with X-gal and IPTG (Sigma). White colonies, indicating positive transformants, were picked and used to inoculate 3 mL Lennox L broth (Sigma)+100 ,μg/mL ampicillin cultures. The plasmids were purified from the cells using a QIAGEN DNA minipreparation kit according to the manufacturer's instructions. The purified plasmids were analyzed by restriction digestion to identify the ones containing the DMBT1 PCR product.

B. pDMBT1-1/luc

The 1.4 kb DMBT1 promoter fragment, from −1347 to +41 (nucleotides 2259-3647 of SEQ ID No:1), was subcloned into pGL3basic (Promega), which is a luciferase reporter vector that lacks eukaryotic promoter or enhancer sequences, as follows; the resulting plasmid was named pDMBT1-1/luc. pDMBT1-1/GEM-T was purified from a 200 mL culture using a QIAfilter maxi kit (QIAGEN) according to the manufacturer's instructions. The 1.4 kb HindIII-MluI DMBT1 fragment was removed from pDMBT1-1/GEM-T by restriction digestion and isolated by electrophoresis on a 1× TAE, 1% SeaKem Gold Agarose Reliant gel, and was purified from the gel using a QIAquick Gel Extraction Kit. The DNA was eluted from the QIAGEN column with 50 μL elution buffer, and then ethanol precipitated. The purified DNA fragment was subsequently ligated to the pGL3basic plasmid that had been digested with HindIII and MluI and purified. The DNA from the ligation reactions was transformed into E. coli and resulting transformants were analyzed as described above. The pDMBT1/1-luc clone was sequenced throughout the entire PCR-derived region in order to verify the sequence of the clone (Lark Technologies).

C. pDMBT1-2/luc

Another plasmid vector carrying a larger portion of the regulatory sequence of the DMBT1 gene was constructed by cloning an upstream region of the human DMBT1 gene from −2921 through +41 (nucleotides 685-3647 of SEQ ID No:1) into the pGL3basic plasmid (Promega). The resulting plasmid was named pDMBT1-2/luc. First, a 5′ portion of the DMBT1 gene from nucleotides −2921 to −926 (nucleotides 685-2680 of SEQ ID No:1) was amplified using PCR. The primers oDMBT1-3: 5′-5′-GAATTCGGTACCGCATTCCACTGGCCTGTTTTATGTTTTGGG-3′-3′ (SEQ ID NO:4), and oDMBT1-4: 5′-GCTTCTTTGCCATGCACTGTTCATCAGTAG-3′ (SEQ ID NO:5) were used. The 2 kb PCR product was ligated to pGEM-T Easy (Promega) as described above to create pDMBT1-2/GEM-T.

The PCR product was removed from the pGEM-T vector by digestion with KpnI and subcloned into KpnI-digested pDMBT1-1/luc, as described above. The resulting subclones were screened for the correct orientation of the 2 kb fragment by digestion with NdeI. The resulting pDMBT1-2/luc construct has a regulatory region consisting of nucleotides −2921 through +41 of the DMBT1 gene.

D. pDMBT1-3/luc

Another plasmid vector carrying a small portion of the regulatory sequence of the DMBT1 gene was constructed by cloning an upstream region of the human DMBT1 gene from −2766 to −2734 (nucleotides 840-872 of SEQ ID No:1) into the pTA-luc plasmid (BD Biosciences Clontech). This promoter region, 5′-CAAGGTCAAGAGATCGACACCATCCTGGCCAAC-3′ (SEQ ID NO:6), is part of an Alu repeat. Oligonucleotides with the sequence of SEQ ID NO: 5 and its complement 5′-GTTGGCCAGGATGGTGTCGATCTCTTGACCTTG-3′ (SEQ ID NO:7) were synthesized (Sigma Genosys). The complementary oligonucleotides were annealed to each other by heating to 96° C. for 3 minutes, and then slow cooling. The annealed oligonucleotides were purified on a 3% BIO-gel agarose gel (BIO101) followed by extraction from the gel with a MERmaid kit (BIO101) according to the manufacturer's instructions. The oligonucleotides were then blunt-end ligated to a pGL3 promoter vector (Promega) that had been digested with Smal and purified as described above for pGL3basic. The ligations were transformed into E. coli strain JM109 and plasmids that were found to contain the appropriately sized inserts were sequenced. One clone having the correct sequence was selected for further use. This plasmid was purified with a QIAfilter Plasmid Maxi Kit, after which the insert was removed by digestion with BglII and MluI and ligated to pTA-luc (BD Biosciences Clontech) that was also digested with BglII and MluI. The resulting plasmid, named pDMBT1-3/luc, was purified with a QIAfilter Plasmid Maxi Kit.

E. pERE-luc

For a positive control, a vector that contained a regulatory region having two tandem estrogen response elements (EREs) from the chicken vitellogenin gene (Klein-Hitpass et al., (1986) Cell 46: 1053-1061) linked to the luciferase reporter gene was constructed. When transformed into an estrogen-responsive cell this vector was able to drive expression of the luciferase gene in response to an estrogen-initiated signal. This vector was named pERE-luc.

The cloning procedure is similar to that for pDMBT1-3/luc with the exception that directional cloning was performed. Oligonucleotide oERE-1:, 5′-GGAGCTAGCTA GAGGTCACAGTGACCTACGAGTCCCTAGAGGTCACAGTGACCTACGAGATCTGGA-3′ (SEQ ID NO:8), which is flanked by two restriction digestion sites NheI and BglII (underlined), was annealed to oERE-2: TCCAGATCTCGTAGGTCACTGTGAC CTCTAGGGACTCGTAGGTCACTGTGACCTCTAGCTAGCTCC (SEQ ID NO:9), purified, and digested with NheI and BglII. The digested, annealed oligonucleotide was cloned into pTA-luc digested with NheI and BglII.

pERE-luc was used as a positive control for estrogen induction of reporter luciferase expression. pTAbasic, pGL3basic, and empty reporter vectors were used as negative controls.

II. Construction of a Vector Expressing a Human Estrogen Receptor

A vector encoding a human estrogen receptor protein was constructed in order to prepare transfected cells expressing a functional estrogen receptor. A full-length (2.0 kb) human estrogen receptor a cDNA was cloned from testis RNA by RT-PCR, using the primers oER-1: 5′-ACGGACCATGACCATGACCCT-3′ (SEQ ID NO:10) and oER-2: 5′-AGCTCTCAGACTGTGGCAGGGAAA-3′ (SEQ ID NO:11). The amplified cDNA was cloned into the TA cloning vector pCR2.1 (Invitrogen Life Technologies, Carlsbad, Calif.). Following sequence verification, it was subcloned into EcoRI-digested pCI-neo (Promega, Madison, Wis.). pCI-neo includes a cytomegalovirus regulatory region, a SV40 poly(A) region, and a neomycin selectable marker. The sequence of pCI-ERα-neo was confirmed by sequence analysis.

III. Construction of Host Cells Expressing a Functional Estrogen Receptor and Having DMBT1-luciferase Reporters

HEK293 human embryonic kidney cells (ATCC) were seeded into 96-well culture plates at 20,000 cells per well in assay medium (DMEM-F12 [Sigma]+10% heat-inactivated charcoal-dextran stripped FBS [Hyclone]+penicillin/streptomycin). Twenty-four to 30 hours later, the cells were co-transfected individually with 10 ng/well of a reporter construct as described in I.A. to I.E. above and with or without (control) 55 ng/well pCI-ERα-neo, using 1 μL per well Lipofectamine2000 (GibcoBRL). Approximately 18 hours after transfection, cells were treated with various concentrations of 17β-estradiol in fresh assay medium for 24 hours.

IV. Luciferase Assay

The luciferase activity of the reporter cell was then assayed using Steady-Glo Luciferase reagent (Promega), according to the manufacturer's instructions. Essentially, 100 μL of Steady-Glo reagent were added directly to each well of transfected cells (described in III) 24 hours after treatment with 17β-estradiol. After a one-hour incubation, the entire supernatant was transferred to white 96-well plates and the Luminoskan Ascent Luminometer with a two- second read time per well. The luciferase activity of estrogen-treated cells co-transfected with each of the reporter constructs and pCI-ERα-neo was normalized to: 1) the luciferase activity of cells which were transfected with the reporter constructs, but not the receptor construct, and 2) cells co-transfected with the reporter constructs and pCI-ERα-neo but not treated with 17β-estradiol.

The results of a cotransfection experiment are shown in FIG. 6. Cells were cotransfected with pCI-ERα-neo and the vectors listed on the right side of the figure. Cells cotransfected with pCI-ERα-neo and the control vectors pTA and pGL3, individually, which are not linked to a regulatory region, produced a very low signal over a wide range of estradiol concentrations. Cells cotransfected with the pCI-ERα-neo and pERE-luc produced a strong signal that was dependent on the concentration of estradiol used. The data provided by these control transfections validate the functionality of the cell-based reporter system.

Cells cotransfected with various DMBT1-regulatory region/luciferase vectors produced the following results. First, cells cotransfected with pCI-ERα-neo and pDMBT1-1luc, which contains −1347 to +41 of the DMBT1 regulatory region (nucleotides 2259-3647 of SEQ ID No:1), was not estrogen-inducible at any of the estradiol concentrations tested. Cells cotransfected with pCI-ERα-neo and pDMBT1-2/luc, which contains −2921 through +41of the DMBT1 regulatory region (nucleotides 685-3647 of SEQ ID No:1), was slightly estrogen-inducible over the range of estradiol concentrations tested. Finally, cells cotransfected with pCI-ERα-neo and pDMBT1/3-luc, which contains a minimal (33 bp) portion of the DMBT1 regulatory region from −2766 to −2734 (nucleotides 840-872 of SEQ ID No:1), was strongly estrogen inducible in a dose-dependent manner (greater than five-fold at a maximal concentration of estrogen).

These results demonstrate that the upstream portion of the DMBT1 regulatory region, particularly the region from −2766 to −2734, is important for estrogen-responsiveness. Surprisingly, the pDMBT1-2/luc vector, which contains nucleotides −2921 through +41 (which includes the −2766 to −2734 region), was not nearly as estrogen-responsive as the pDMBT1-3/luc vector (which includes only the −2766 to −2734 region). Although not wishing to be bound by theory, it is possible that one more elements are present in the region of the DMBT1 regulatory region upstream of −2766, and/or downstream of −2734, that interfere with estrogen-responsiveness via the Alu sequence in at least this type of cell-based system.

This example illustrates the construction and usefulness of new cell-based systems expressing a functional estrogen receptor and a vector including a DMBT1 regulatory sequence operably linked to a reporter sequence. More particularly, it demonstrates new cell-based systems having a vector encoding a functional estrogen receptor and a vector including a DMBT1 regulatory sequence operably linked to a reporter sequence.

This example also illustrates the construction and usefulness of new isolated nucleic acid molecules that have a DMBT1 regulatory region that is operably linked to a reporter sequence, where the nucleic acid can be responsive to estrogen-induced signals in a cell system. Useful nucleic acids include the sequence −2766 to −2734 of the DMBT1 regulatory (nucleotides 840-872 of SEQ ID No:1). Other useful nucleic acids include a DMBT1 regulatory region upstream of −2734, and upstream of −1347, including sequence −2766 to −2734 (nucleotides 840-872 of SEQ ID No:1), and that is operably linked to a reporter sequence.

### EXAMPLE 5

### Effects of Progesterone Receptor Modulators (PRMs) on Monkey Endometrial Tissue

As a first step in investigating effects on DMBT1 expression of PRMs, studies were conducted to examine gene expression in endometrial tissue of OVX monkeys that were co-treated with estrogen and a PRM. OVX monkeys co-treated with estrogen and a PRM generally showed a significant decrease in endometrial thickness after a period of treatment, relative to estrogen alone.

I. Animals And Drug Administration

Animals and treatment were the same as Example 1 with the following exceptions. To study the effect of PRMs on the endometrium and gene expression, groups of three ovariectomized (OVX) monkeys were given an estrogen implant (E2), plus daily oral doses of the following PRMs for 19 days: mifepristone at 10 mg/kg/d; 17N11-DE (17β-N-substituted-11β-dimethylaminophenyl-estra-4,9-dien-3-one) 1.0, 3.0, or 10 mg/kg/d; 17N11-PE (17β-N-substituted-11β-piperidinylphenyl-estra-4,9-dien-3-one) at 1.0, 3.0, or 10 mg/kg/d. A placebo (0.5% methylcellulose) was administered as a control. 17N11-DE and 17N11-PE are two PRMs that have been described in PCT Publication No. WO 99/62928. Tissue samples were taken from endometria and myometria for histological analysis.

Tissue samples were also taken from endometria for subsequent gene expression studies as described in Example 5. Biopsies were placed in a sterile container and snap frozen immediately in liquid nitrogen. The biopsies were then transferred to a −80° C. freezer and stored until required.

II. Results

The average endometrial thicknesses in treated and control OVX monkeys are shown in Table 1. In animals lacking estrogen, whether they were ovariectomized or received leuprolide for 40 days, the endometrium is atrophic, with a thin to nonexistent stromal layer. Referring to Table 1, the thickness of the endometrium in the ovariectomized group (A) is about three times smaller than that of the leuprolide-treated endometrium (B). This result may be explained by differences in residual serum hormone levels between the two groups, with the leuprolide-treated group having two to three times higher estradiol levels than the ovariectomized group (20 to 30 pg/mL versus approximately 10 pg/mL, respectively) throughout the study. Following 19 days of treatment with estrogen, the typical hyper-proliferative effect of the hormone was observed: the stromal layer thickens dramatically, and there is nascent secretory gland formation. Averaging the data for the three monkeys in each group, the estrogen-stimulated endometrium (C) is seven to eight times thicker than it is in the ovariectomized control (A), and 2.5 times thicker than the chemically castrated endometrium (B).

When a PRM (for example, mifepristone, 17N11-DE, or 17N11-PE) is co-administered at a high (maximally efficacious) dose, the endometrium shrinks to about the thickness of that seen with leuprolide-treated tissue (Table 1). There is some secretory gland formation, but the glands appear flat and disorganized. At lower doses, endometrial thickness appears to be reduced more by 17N11-DE than by 17N11-PE (Table 1).

TABLE 1
Average Endometrial Thickness in Treated Monkeys*

| Treatment | | Endometrial Thickness (mm) |
|---|---|---|
| (A) | Ovx/Placebo/Vehicle | 0.400 |
| (B) | Intact/leuprolide | 1.25 |
| (C) | Ovx/E2/Vehicle | 3.03 |
| (D) | Ovx/E2/Mif (10 mg/kg) | 1.17 |
| (E) | Ovx/E2/17N11-DE (1.0 mg/kg) | 1.37^{†} |
| (F) | Ovx/E2/17N11-DE (3.0 mg/kg) | 1.17 |
| (G) | Ovx/E2/17N11-DE (10 mg/kg) | 1.34 |
| (H) | Ovx/E2/17N11-PE (1.0 mg/kg) | 2.10 |
| (I) | Ovx/E2/17N11-PE (3.0 mg/kg) | 1.50 |
| (J) | Ovx/E2/17N11-PE (10 mg/kg) | 1.00 |

*Mean of data from three animals, except where indicated.
^{†}Average of two animals. No endometrium present in third animal.

### EXAMPLE 6

### PRMs Inhibit DMBT1 Gene Expression in Estrogen/PRM-Treated Monkey Endometrium

Tissue samples were taken from endometria of the ovariectomized monkeys cotreated with estrogen and PRMs or a placebo, as described in Example 5. Preparation of total RNA from these samples, probe preparation, and DNA microarray hybridization were carried following a procedure exactly as that described in Example 1.

Using two-fold regulation across all experimental identifiers as a cut-off point, relative to estrogen alone, the expression of 164 gene identifiers was altered in the presence of 10 mg/kg/d mifepristone, 163 in the presence of 10 mg/kg/d 17N11-PE, and 467 in the presence of 10 mg/kg/d 17N11-DE. Most of the additional genes that were altered by 17N11-DE were regulated less than 2.5-fold.

PRMs inhibited the induction of DMBT1 gene expression by estrogen in a dose-dependent manner. Referring to FIG. 7, microarray analysis revealed that estrogen-induced DMBT1 mRNA levels were repressed about 20-fold by 10 mg/kg mifepristone, about 5-fold by 1 mg/kg 17N11-DE, about 25-fold by 3 mg/kg 17N11-DE, about 2-fold by 1 mg/kg 17N11-PE and about 4-fold by 3 mg/kg 17N11-PE.

According to these data, DMBT1 gene expression was downregulated in the endometrium in response to PRMs when coadministered with estrogen.

### EXAMPLE 7

### A DMBT1 Sequence-Regulated Cell-Based Assay Useful for Progesterone Agonist (and PRM) Identification

A progesterone- and estrogen-responsive cell carrying a nucleic acid having a DMBT1 regulatory sequence linked to a reporter sequence is constructed and used to screen for test compounds having progesterone antagonist activity, such as PRMs. As demonstrated in Example 4, cells transfected with the ER and DMBT1-reporter vectors were able to produce a signal when estradiol was applied to the cells. A vector expressing a functional progesterone receptor is transfected into these cells and used to identify test compounds that modulate the ER-promoted signal through the progesterone receptor.

I. DBMTl/Luciferase Reporter Vector

Vectors as described in Example 4 are introduced into a suitable cell type for the assay. Particularly useful reporter vectors include the Alu sequence of the DMBT1 regulatory region fused to a luciferase gene, such as pDMBT1-3/luc. Other reporter vectors can be constructed and used, for example, vectors that contain DMBT1 regulatory sequences, or a portion thereof, that are operationally linked to other reporter genes such as chloramphenicol acetyltransferase or galactosidase.

II. Estrogen and Progesterone Receptor Vectors

The activity of a PRM is assessed using a cell expressing functional estrogen and progesterone receptors. Vectors encoding a functional estrogen receptor, such as pCI-ERα-neo, which have been described in Example 4, are introduced into a suitable cell type for the assay.

Similarly, a vector encoding a functional progesterone receptor (PR) is constructed and introduced into the same cell as the ER vector. Vectors for the expression of PR have previously been constructed and are described in, for example, Conneely et al. (1987) Biochem. Biophys. Res. Commun. 149:493-501. The PR receptor is cloned into a vector allowing expression in eukaryotic cells, such as mammalian cells. The PR vector can include enhancer and promoter sequences that drive the expression of PR, a polyadenylation signal sequence, and selectable markers for maintenance in cells, such as a neomycin gene. Vectors containing these elements are commercially available from, for example, Promega (Madison, Wis.), and known techniques allow for cloning the PR gene into a suitable expression vector in any desired manner.

In some cases both the ER and PR genes are placed on one expression vector and introduced into a cell for expression of these genes.

As an alternative to introducing vectors having ER and PR genes, a single vector carrying either the ER or the PR gene is introduced into a cell that provides endogenous expression of PR or ER, respectively. Suitable cell types are detailed below.

III. Cells Expressing Endogenous ER or PR

The DMBT1/luciferase reporter vector and the PR- or ER-expression vector are introduced into a cell line providing endogenous expression of ER or PR, respectively. The reporter vector and ER vector is introduced into the breast cell line, T47D (Horwitz, K.B. et al. (1982) Cell 28:633), which expresses PR (and low levels of ER), or the reporter vector and the PR vector is introduced into the breast cell line, MCF7 (Shupnik M A et al. (1989) Mol. Endocrinol. 3:660) which expresses ER. Alternatively, and as indicated above, the reporter, ER, and PR genes are all introduced into a suitable cell line.

The vectors are introduced into the cells using methods known to those skilled in the art. These methods include, but are not limited to, calcium phosphate precipitation and electroporation. Commercially available kits and devices are available to perform these techniques such as the CellPhect™ Transfection kit from Pharmacia (Piscataway N.J.), and the Cellporator and Lipofectin™ from Invitrogen Life Technologies (Gaithersburg, Md.). Following introduction of the vectors into the cells by any of these methods the cells can be selected based on a selectable marker, constitutive reporter production, induction of reporter expression by estrogen, inhibition of estrogen induction by PRMs, satisfactory attachment to microtiter plates used in the high-throughput screen, and acceptable standard deviation in multiple estrogen induction reporter expression assays. Clones that satisfy the necessary criteria for a high throughput screen are then selected for use.

As an alternative to generating stable clones, transient transfection is used. Transfected cells would be used for assay 12 to 24 hr later.

IV. Bioluminescence Assay

To assay for luciferase expression in transfected cells, cells are incubated with various concentrations of estrogen, in the presence or absence of a test compound suspected of having anti-progestogenic activity. The treated cells are then assayed according to the procedure used in Example 4. Specifically, cells are lysed in a buffer containing lysis reagents and a luciferase substrate such as Steady-Glo™ (Promega, Madison, Wis.) or LucLite™ Plus (Packard, Meriden Conn.). Lysates are incubated at room temperature. Measurement is performed within two hours of lysis. Photons are detected using a light-sensitive instrument, such as a luminometer (for example, Luminoscan Ascent (ThermoLab Systems, Franklin, Mass.)) or scintillation counter (for example, TopCount (Packard)). Typically, each well is read for 0.1 to 2 seconds.

V. High-Throughput Screening

Luciferase reporter cells (clones or transiently transfected) are first washed with PBS, harvested, and counted using a hemocytometer and the Trypan Blue exclusion method, according to protocols provided by Sigma Chemical Company, St. Louis, Mo. Cells are then diluted into media, and 0.2 mL of cell suspension is plated into each well of a 96-well microtiter plate using methods known to those skilled in the art. Twelve to 24 hr later, estrogen (at a final concentration of 0.1 to 10 nM) is added to cells on the microtiter plate. Also added is the test compound or an equal volume of the solvent in which the test compound is dissolved. Therefore, each test well on the assay plate contains luciferase reporter cells, estrogen, and the test compound, while each control well on the assay plate contains luciferase reporter cells, estrogen, and the solvent control. An additional set of control wells contains only reporter cells and the solvent control. The assay plate is incubated for 12 to 48 hours at 37° C., in 5% CO₂. The cells are then lysed and luminescence is detected as described above. Data are processed. The bioluminescent signal detected in the test well is compared with that of the estrogen-containing control well. A test compound with anti-progestogenic activity will result in a decreased bioluminescent signal compared to the latter control.

While the foregoing specification teaches the principles of the present invention, with examples provided for the purpose of illustration, it will be understood that the practice of the invention encompasses all of the usual variations, adaptations and/or modifications as come within the scope of the following claims and their equivalents. References disclosed in this text are incorporated by reference herein.

### EXAMPLE 8

### Effects of Progestin on Rat Endometrial Tissue

Six weeks old ovariectomized Wistar rats (Charles River, Wilmington, Mass.) were dosed for either one day or six weeks with vehicle or test compounds. All treatments were delivered orally, once daily, in 0.5% methylcellulose or sesame oil. After treatment, the uteri were removed, total RNA was purified from the tissues using TRIzol reagent (Invitrogen, Carlsbad, Calif.) as directed by the manufacturer.

Quantitative reverse transcription-polymerase chain reactions (RT-PCR) of DMBT1 from rat RNA samples were carried out using a One-Step RT-PCR Master Mix Kit (Applied Biosystems, Foster City, Calif.) and an ABI Prism 7000 Sequence Detection System (Applied Biosystems), as described by the manufacturer. Commercially available primers and FAM-labeled MGB probes for human and rat DMBT1 (catalog numbers Hs00244827_ml and Rn00575705_ml, respectively) were obtained from Applied Biosystems (Foster City, Calif.). Messenger RNA levels were normalized against 18S ribosomal RNA levels determined using primers and a VIC- and TAMRA-labeled probe from the same vendor.

The results as assessed by quantitative PCR is shown in FIG. 3B. Ethinyl estradiol treatment for one day (RNA preparation approximately 24 hr after a single dose) resulted in a significant increase in DMBT1 mRNA levels. The induction was maximal (six-fold) at a dose of 0.3 mg/kg of the estradiol. Co-treatment with a progestin (medroxyprogesterone acetate) blocked the increase in mRNA levels in a dose-dependent manner, an effect that was blocked in turn by 10 mg/kg of the progestin antagonist, mifepristone.

### EXAMPLE 9

### Immunohistochemical Analysis of Uterine Expression

Mature, six week-old ovariectomized or sham-operated Wistar rats (Charles River) were dosed with ethinyl estradiol, tamoxifen or raloxifene for six weeks. Uteri were removed, sectioned for immunohistochemical analysis for DMBT1 expression using a polyclonal goat antibody against the human protein.

Tissues were trimmed and processed for paraffin embedding according to conventional methods. Five-micron sections were cut, mounted onto SuperFrost Plus+(Fisher Scientific, Pittsburgh, Pa.) microscopic slides and dried overnight. The protocols for routine single immunohistochemistry (IHC) have been described previously (D'Andrea et al., 2001, Biotechnic Histochem. 76:97-106). Briefly, tissue sections on microscopic slides were dewaxed and re-hydrated. Slides were microwaved for 5 min in Target buffer (Dako, Carpenturia, Calif.), cooled, placed in phosphate-buffered saline (PBS, pH 7.4) and treated with 3% (v/v) H₂O₂ for 10 min at room temperature. All incubations (30 min each) and washes were performed at room temperature. Normal rabbit blocking serum (Vector Labs, Burlingame, Calif.) was placed on all slides for 10 min. After a brief rinse in PBS, sections were treated with goat polyclonal DMBT1 primary antibody (1:25, Hypromatrix, Worcester, Mass.). Slides were then washed in PBS and treated with rabbit anti-goat biotinylated secondary antibodies (Vector Labs). After washing in PBS, the avidin-biotin-horseradish peroxidase complex reagent (HRP, Vector Labs) was added. All slides were washed and treated with 3,3′-diaminobenzidine (DAB, Biomeda, Foster City, Calif.) two times for 5 min each, rinsed in distilled water, and counterstained with hematoxylin.

Protocols for double IHC have been described previously (D'Andrea et al., 2001, supra). The rabbit polyclonal PCNA (proliferating cell nuclear antigen) antibody (1:4000; Sigma, St. Louis, Mo.) was detected using the HRP:SG detection system as described for routine single IHC, except the use of the SG chromogen (Vector Labs). The second primary antibody, specific for DMBT1, was similarly incubated on the tissues. After washes, the biotinylated secondary goat anti-goat antibody (Vector Labs) was placed on the slides for 30 min at room temperature. Subsequently, sections were treated with the alkaline phosphatase ABC reagent (AP-ABC, Vector Labs) for 30 min. DMBT1 was then detected using the Fast Red chromogen (Sigma, St. Louis, Mo.). Slides were dipped in hematoxylin and cover slipped with a water-based mounting medium (Dako). Negative controls included: 1) replacement of each primary antibody with non-immune serum, 2) omission of each primary antibody, and 3) double negative controls.

It was found that, compared to the sham (panel A) and the ovariectomized control, estrogen treatment produced the expected thickening of the luminal epithelium, which was even more pronounced following tamoxifen treatment, but not with raloxifene treatment (data not shown). This effect on the epithelium correlated with gross effects on uterine weight (FIG. 1). DMBT1 staining was restricted to the luminal epithelium, with little or no staining in glandular epithelia or in stromal cells. However, staining in the luminal epithelium was variable, with some cells intensely stained, others weakly stained, and others not stained at all. Raloxifene and especially tamoxifen appeared to produce higher cell levels of DMBT1 than estrogen. Staining was extra-nuclear in all cases. In sham-operated and ovariectomized, estrogen-treated uterine epithelium, staining was predominantly at the luminal aspect of the cell. In contrast, staining was uniformly distributed throughout positive cells in epithelium from SERM-treated uteri.

To determine if a difference between those cells that expressed DMBT1 protein and those that did not lay in their proliferative status, slides were co-stained with the DMBT1 antibody and a PCNA antibody. While many DMBT1-expressing cells also stained for PCNA, there was not an absolute overlap (data not shown), indicating that the variability of DMBT1 expression is due to more than the proliferative state of the cell.

TABLE 2

SEQ ID NO. 1

| | |
|---|---|
| gggggagtga gtgaaggang gtaggaaggg gtccccttat aacccaattg ggaagaccgg | 60 |
| ctctctgagc aacaaaaatg agcgacatta taaatatcan acatacaatg tcaaaagatt | 120 |
| gttacaacag taacaatgac cggcaattat ggtgtgctac tcaatgaggg taaaattaga | 180 |
| gtttggggtc agactgacac ctgaagggga gccctggtca cacagattac nggattcagg | 240 |
| gactggaagg caaacagcag cagccggaag gtgctacagc cccgaccctt cccaccaggc | 300 |
| tccccgtcct tctgtccccc tcgtctgtca ggtcacaggg ctcctggctc agaaatcaga | 360 |
| gacccctcag aggtgggagg tgggaggggc cctgggctgc ctccctgatc tccgtcttcc | 420 |
| tctggcttcc cagggcactg gtctgtgggt agaaagaatc tttagaagaa ggagaacaac | 480 |
| gggctgtttt gggaagtctg cctgggaccc cagctcagct tcgtggcagg gatgtgtgcc | 540 |
| tggcagcccc aggggccctg ctgggcctct cagtgccctg tcttatgaag ggaggtcttg | 600 |
| gcttcccaca ggtaatcctc agctctgacg gcaggtggcc ctaggggagg ggcctggaag | 660 |
| aggactccct caccacagtc aggagcattc cactggcctg ttttatgttt tggggcttcc | 720 |
| ccatgaattc caatttagga aaggattctg tagctttaaa aaaaaaagcc atacatggcc | 780 |
| gggcgtggtg actcatgcct gtaatctcag aactttgaga ggccgaggca ggcagatcgc | 840 |
| aaggtcaaga gatcgacacc atcctggccg acatggtgaa accctgtctc tactaaaaat | 900 |
| acaaaagtta gctgggtgtg gtgatgcacg cctgtagttc cagctactcg ggaggctgag | 960 |
| gcaggagaat cgcttgaact cgggaggcgg aggttgtagt gagccgagat tgcaccactg | 1020 |
| cactccagcc tgggcgacag aacaagactc cgtcttaaaa taaataagta aataaataaa | 1080 |
| taaatggcca catatgatcc agggggctct tccccataac gaccagtcct ctggtgctgg | 1140 |
| gatttagggg cttggtccca cctcctggag tgtgctgtgg gggtgccgct ggcttaattg | 1200 |
| cctggggaag gtgctttgca aatggggccc gggcgccgag tctctgagct ttaatgggct | 1260 |
| tatcttgctt cctgttgttc ttcatggcag ttctggggtg aataaaatgg aactttgtgg | 1320 |
| ttgtggtctt ccaacccctg ctgtttaagc agatgcttct aaccagtnng aaggggaaga | 1380 |
| tgagggtcct ctggcagggg ctgctgctgg agaaggttgt ccccctgaac ccttctcttc | 1440 |
| gtccaaggaa agacctccct aaatggtgtc ttatgagcca caaaatggcc ttgggacaag | 1500 |
| atgaggcacc tgacggtgaa ggctgaggtc ccacacggtg ttggcaggtg gccctcaagc | 1560 |
| ccattgtctt ctgcccgcat gcccaggacc gccacatcct tcctgttcca ctgacgtcac | 1620 |
| ccatttccca cctgggatgc ctacctgggc tggctttgaa atgacagcta ggcttcacca | 1680 |
| cttccttctt cctggtgtct cctgaacaaa aagctcacaa acccttattg tctcaggatc | 1740 |
| ttttctgcta actaccatgg gcaaaagatg cagtcaaaac aacaaaaacg gggaggttac | 1800 |
| gcagttcaga aaatcccaca cattctcaag aagggtgtcc ccgccaagct gagagatacc | 1860 |
| tgggacatca tgctttcctg gaggggaagg ctccctgccc ccattcctgt acagccggga | 1920 |
| gagtcgctcc tgttcgtcat cccatcttct cgctctcatt tgctgggctg acctctgctg | 1980 |
| gtatttcaag actgtttggt gccaccctct ctgagagatg gcctccctca tctgatttag | 2040 |
| atgcttttct ttaccttctc atagcagctt gtactaatac ttgctacccc ctgttgaaat | 2100 |
| tgtcaccagg caggtctgtc tctaagacca gacagtctgc ttttgtcttg gaaggacact | 2160 |
| tattacccna ncntgttcac aggtatttag tattcttacc atcatctttc cctgctgtgc | 2220 |
| tcccggcaga caattctaat ctgtcatgac actctgatga tcccagaccc agctgcatta | 2280 |
| tcattctcag tccaacactc caggaaccaa gggatcacaa tccccttcta agaggaatcc | 2340 |
| agcatgtgcc tggtcttggg cattccctgg tangtgagta accctgttct ctcgtcaccc | 2400 |
| agtgcttatc agttgctgat ctggcagtag gaggatgaaa cacagtgagc ctattctgtg | 2460 |
| ttcctattct actcaagggg tgaagaggca cctggaaaca acaggaagag ttgtaggatt | 2520 |
| aaaaaggaca tccaagattg aatgtaactt tcatctggat gaagccaaag gcagacttcc | 2580 |
| agccctaaat tctgactggt ggctgacaca ggacatgggt tcatggtacc cttctagaat | 2640 |
| gcagcataga ctactgatga acagtgcatg gcaaagaagc caagtgtcat ttcatggcct | 2700 |
| cagcctctca gctgagaagc agggcacagc tcacccaggc taggaaaaac agaggcaagt | 2760 |
| cctggaaagc tgtctgcttt taaccaagag ttactggcca tcaagtgtct tggttaaaaa | 2820 |
| ataagtgtca ggcaaccttc ttggtagata gagtgtgttg ggggcgatta tcagagtctg | 2880 |
| gtaatgactt ctgagggtcc caaagagtga agtgatattt acatagcaaa tccaaggang | 2940 |
| gggattgtgt gcaatatang tggangtggg ggcaggtttt gtgggtttgc caagctccaa | 3000 |
| ggtcatacaa tgtgcatgtc aaggacaaga aatcaaagcc atgtgaaatg gttggaggtg | 3060 |
| gttcagtttg aggtcatgtg tttctcagct cctgttgtgg aattagtgtg agacccagaa | 3120 |
| gactgtggcc aaagctatta tggacccatg gtctctgtgg actcatccct catgccttct | 3180 |
| gctctctgat cacatccaca ctcatgtcat cctcgttctt ccaaggtgag gttactagca | 3240 |
| ctgcacaggg gctgatgaga gcatgtcctg ccaggaaaaa ccatcccaaa gagatgcttt | 3300 |
| cccccttggc actgtgtcct gtatttgctc agcagcccac atcctgttct gccccaaacc | 3360 |
| ttggggcaga cttcccacag gtgaatttga actccccaag attaaaatca agcctgtatt | 3420 |
| caggaaacac ttgggagtcc tcgaggttca ccgagaggga agttggaaat ttttcactta | 3480 |
| tgtcagtgcg tttgcagttg ggcaacagcc agattgttca tatggcaatc aatcaaacac | 3540 |
| acctaagttt tttccacata ttagccatcg actgttagca aaagccctca cttcctttat | 3600 |
| attgatttat agcagcagca gaaatatacc accctagagg acacacctcc ttttagctag | 3660 |
| gtacctataa atgtccagga ttttctattc aattgagaag aacccagcaa aatgg | 3715 |

SEQ ID NO. 2

gaattcacgc gtgatcccag acccagctgc attatcattc tc

SEQ ID NO. 3

gaattcaagc ttggtgtgtc ctctagggtg gtatatttct gc

SEQ ID NO. 4

gaattcggta ccgcattcca ctggcctgtt ttatgttttg gg

SEQ ID NO. 5

gcttctttgc catgcactgt tcatcagtag

SEQ ID NO. 6

caaggtcaag agatcgacac catcctggcc aac

SEQ ID NO. 7

gttggccagg atggtgtcga tctcttgacc ttg

SEQ ID NO. 8

ggagctagct agaggtcaca gtgacctacg agtccctaga ggtcacagtg acctacgaga

SEQ ID NO. 9

tccagatctc gtaggtcact gtgacctcta gggactcgta ggtcactgtg acctctagct
agctcc

SEQ ID NO. 10

acggaccatg accatgaccc t

SEQ ID NO. 11

agctctcaga ctgtggcagg gaaa

1. A method comprising steps of:
(a) contacting an estrogen-responsive system with a test compound;
(b) determining gene expression from a gene operably linked to a DMBT1-regulatory sequence in the estrogen-responsive system; and
(c) detecting estrogenic activity as a result of the test compound.

2. The method of claim 1 wherein step (c) comprises measuring gene expression relative to a control and identifying test compounds that result in increased gene expression and therefore have estrogenic activity.

3. The method of claim 1 wherein the gene is DMBT1.

4. The method of claim 3 wherein the determining step comprises measuring an amount of a DMBT1 nucleic acid.

5. The method of claim 3 wherein the measuring step comprises measuring DMBT1 protein.

6. The method of claim 1 wherein the estrogen-responsive system comprises uterine tissue.

7. The method of claim 1 wherein the estrogen-responsive system comprises mammary tissue.

8. The method of claim 1 wherein the estrogen-responsive system comprises osteoid tissue.

9. The method of claim 1 wherein the estrogen-responsive system is an animal.

10. The method of claim 9 wherein the animal is a primate.

11. The method of claim 10 wherein the primate is a monkey.

12. The method of claim 9 wherein the animal is a rodent.

13. The method of claim 9 wherein the animal has been treated to reduce systemic levels of estrogen.

14. The method of claim 1 wherein the estrogen-responsive system comprises a cell.

15. The method of claim 14 wherein the cell comprises an estrogen receptor.

16. The method of claim 15 wherein the cell comprises a reporter gene operably linked to the DMBT1 regulatory sequence.

17. The method of claim 16 wherein the reporter gene encodes a protein selected from the group consisting of green fluorescent protein (GFP), β-galactosidase (lacZ), luciferase (luc), chloramphenicol acetyltransferase (cat), β-glucuronidase, neomycin phosphotransferase, and guanine xanthine phosphoribosyl-transferase.

18. The method of claim 2 wherein the control is an estrogen-responsive system not contacted with the test compound.

19. The method of claim 2 wherein the control comprises cells and tissues nonresponsive to estrogen.

20. The method of claim 1 wherein step (c) comprises detecting test compound stimulating no estrogenic activity or anti-estrogenic activity.

21. A method comprising the steps of:
(a) contacting a first estrogen-responsive system with a test compound;
(b) contacting a second estrogen-responsive system with the test compound;
(c) measuring expression of a gene controlled by a DMBT1-regulatory sequence from the first estrogen-responsive system and measuring expression of a gene controlled by a DMBT1-regulatory sequence from the second estrogen-responsive system; and
(d) correlating (i) an increase in expression in the first estrogen-responsive system and a decrease or no detectable expression in the second estrogen-responsive system, or (ii) a decrease or no detectable expression in the first estrogen-responsive system and an increase in expression in the second estrogen-responsive system with a selective estrogenic activity.

22. A method comprising the steps of:
(a) contacting a first estrogen-responsive system with a test compound;
(b) contacting a second estrogen-responsive system with the test compound;
(c) measuring a first estrogenic effect comprising expression of a gene controlled by a DMBT1-regulatory sequence in the first estrogen-responsive system;
(d) measuring a second estrogenic effect from the second estrogen-responsive system; and
(e) correlating (i) an increase in expression from the first estrogen-responsive system and no estrogenic effect or an anti-estrogenic effect from the second estrogen-responsive system, or (ii) a decrease or no expression from the first estrogen-responsive system and the presence of an estrogenic effect from the second estrogen-responsive system with a selective estrogenic activity.

23. The method of claim 22 wherein the first estrogen-responsive system is an estrogen-responsive tissue or cell from an animal and the second estrogen-responsive system is different than the first estrogen-responsive system.

24. The method of claim 22 wherein the first estrogen-responsive system comprises a cell having a nucleic acid having a DMBT1 regulatory sequence operably linked to a reporter gene and the second estrogen-responsive system comprises an estrogen-responsive tissue or cell from an animal.

25. The method of claim 22 wherein measuring another estrogenic effect from the second estrogen-responsive system comprises measuring cell proliferation or tissue size.

26. The method of claim 22 wherein measuring another estrogenic effect from the second estrogen-responsive system comprises measuring the amount of a component produced from the second estrogen-responsive system.

27. A method comprising the steps of:
(a) contacting an estrogen- and progesterone-responsive system with an estrogenic compound;
(b) contacting the estrogen- and progesterone-responsive responsive system with a test compound;
(c) obtaining information indicative of expression of a gene controlled by a DMBT1-regulatory sequence from the estrogen- and progesterone-responsive system relative to a control; and
(d) using the information obtained in step (c) to determine a progestogenic activity or an anti-progestogenic activity.

28. The method of claim 27 wherein step (c) comprises measuring expression of the gene relative to a control and step (d) comprises correlating a decrease in the expression with a progestogenic activity or an anti-progestogenic activity.

29. The method of claim 27 wherein the estrogen- and progesterone-responsive system comprises a functional estrogen receptor and a functional progesterone receptor.

30. The method of claim 27 wherein the test compound having anti-progestogenic activity is a progesterone receptor modulator.

31. The method of claim 27, further comprising a step of measuring a progesterone-responsive effect that is different from the expression of a gene controlled by a DMBT1-regulatory sequence.

32. A method comprising the steps of:
(a) treating a subject with a compound capable of stimulating expression of DMBT1 expression in the subject;
(b) detecting DMBT1 expression in the subject as a marker of an estrogenic effect.

32. The method of claim 32 wherein step (b) comprises measuring expression of DMBT1 from the subject relative to a control.

33. The method of claim 32 wherein step (b) comprises obtaining a biological sample from the subject and measuring DMBT1 expression in the biological sample.

34. An estrogen-responsive system comprising a nucleic acid having a DMBT1 regulatory sequence operably linked to a reporter gene.

35. The system of claim 34 wherein the estrogen-responsive system comprises a cell having an estrogen receptor.

36. The system of claim 35 wherein the cell is selected from the group consisting of Ishikawa, MCF-7, MG63, HUVEC, and SK-N-MC cells.

37. The system of claim 36 wherein the reporter gene encodes a protein selected from the group consisting of green fluorescent protein (GFP), β-galactosidase (lacZ), luciferase (luc), chloramphenicol acetyltransferase (cat), β-glucuronidase, neomycin phosphotransferase, and guanine xanthine phosphoribosyl-transferase.

38. The system of claim 37 wherein the reporter gene encodes luciferase.

39. The system of claim 34 wherein the DMBT1 regulatory sequence comprises nucleotides 840-872 of SEQ ID NO.1.

40. The system of claim 39 wherein the DMBT1 regulatory sequence consists of at least 50 consecutive nucleotides from nucleotides 1-2259 of SEQ ID NO:1.

41. An isolated nucleic acid comprising a DMBT1 regulatory sequence operably linked to a reporter gene, wherein the DMBT1 regulatory comprises nucleotides 840-872 of SEQ ID NO.1.

42. The isolated nucleic acid sequence of claim 41, wherein the DMBT1 regulatory sequence is capable of directing expression of a gene operably linked thereto and has at least 85% sequence identify to any consecutive 30-mer within SEQ ID NO:1.

43. The isolated nucleic acid sequence of claim 41 wherein the DMBT1 regulatory sequence consists of nucleotides 1-2259 of SEQ ID NO:1.

44. The isolated nucleic acid sequence of claim 41 wherein the DMBT1 regulatory sequence consists of nucleotides 840-872 of SEQ ID NO.1.

45. A method for reducing the upregulation of DMBT1 comprising the step of administering a compound with anti-progesterone activity and a compound with estrogenic activity.

46. A method to identify compounds with estrogenic activity comprising screening for upregulation of DMBT1 gene expression.

47. A method to identify compounds with antiprogesterone activity comprising screening for compounds that produce a decrease in DMBT1 expression.


SEQUENCE LISTING (11 sequences)

SEQ ID NO: 1 - DNA, 3715 bp, Homo sapiens
  misc_feature (19)..(19): n is a, c, g, or t
gggggagtgagtgaagganggtaggaaggggtccccttataacccaattgggaagaccggctctctgagcaacaaaaatgagcgacattataaatatcanacatacaatgtcaaaagattgttacaacagtaacaatgaccggcaattatggtgtgctactcaatgagggtaaaattagagtttggggtcagactgacacctgaaggggagccctggtcacacagattacnggattcagggactggaaggcaaacagcagcagccggaaggtgctacagccccgacccttcccaccaggctccccgtccttctgtccccctcgtctgtcaggtcacagggctcctggctcagaaatcagagacccctcagaggtgggaggtgggaggggccctgggctgcctccctgatctccgtcttcctctggcttcccagggcactggtctgtgggtagaaagaatctttagaagaaggagaacaacgggctgttttgggaagtctgcctgggaccccagctcagcttcgtggcagggatgtgtgcctggcagccccaggggccctgctgggcctctcagtgccctgtcttatgaagggaggtcttggcttcccacaggtaatcctcagctctgacggcaggtggccctaggggaggggcctggaagaggactccctcaccacagtcaggagcattccactggcctgttttatgttttggggcttccccatgaattccaatttaggaaaggattctgtagctttaaaaaaaaaagccatacatggccgggcgtggtgactcatgcctgtaatctcagaactttgagaggccgaggcaggcagatcgcaaggtcaagagatcgacaccatcctggccgacatggtgaaaccctgtctctactaaaaatacaaaagttagctgggtgtggtgatgcacgcctgtagttccagctactcgggaggctgaggcaggagaatcgcttgaactcgggaggcggaggttgtagtgagccgagattgcaccactgcactccagcctgggcgacagaacaagactccgtcttaaaataaataagtaaataaataaataaatggccacatatgatccagggggctcttccccataacgaccagtcctctggtgctgggatttaggggcttggtcccacctcctggagtgtgctgtgggggtgccgctggcttaattgcctggggaaggtgctttgcaaatggggcccgggcgccgagtctctgagctttaatgggcttatcttgcttcctgttgttcttcatggcagttctggggtgaataaaatggaactttgtggttgtggtcttccaacccctgctgtttaagcagatgcttctaaccagtnngaaggggaagatgagggtcctctggcaggggctgctgctggagaaggttgtccccctgaacccttctcttcgtccaaggaaagacctccctaaatggtgtcttatgagccacaaaatggccttgggacaagatgaggcacctgacggtgaaggctgaggtcccacacggtgttggcaggtggccctcaagcccattgtcttctgcccgcatgcccaggaccgccacatccttcctgttccactgacgtcacccatttcccacctgggatgcctacctgggctggctttgaaatgacagctaggcttcaccacttccttcttcctggtgtctcctgaacaaaaagctcacaaacccttattgtctcaggatcttttctgctaactaccatgggcaaaagatgcagtcaaaacaacaaaaacggggaggttacgcagttcagaaaatcccacacattctcaagaagggtgtccccgccaagctgagagatacctgggacatcatgctttcctggaggggaaggctccctgcccccattcctgtacagccgggagagtcgctcctgttcgtcatcccatcttctcgctctcatttgctgggctgacctctgctggtatttcaagactgtttggtgccaccctctctgagagatggcctccctcatctgatttagatgcttttctttaccttctcatagcagcttgtactaatacttgctaccccctgttgaaattgtcaccaggcaggtctgtctctaagaccagacagtctgcttttgtcttggaaggacacttattacccnancntgttcacaggtatttagtattcttaccatcatctttccctgctgtgctcccggcagacaattctaatctgtcatgacactctgatgatcccagacccagctgcattatcattctcagtccaacactccaggaaccaagggatcacaatccccttctaagaggaatccagcatgtgcctggtcttgggcattccctggtangtgagtaaccctgttctctcgtcacccagtgcttatcagttgctgatctggcagtaggaggatgaaacacagtgagcctattctgtgttcctattctactcaaggggtgaagaggcacctggaaacaacaggaagagttgtaggattaaaaaggacatccaagattgaatgtaactttcatctggatgaagccaaaggcagacttccagccctaaattctgactggtggctgacacaggacatgggttcatggtacccttctagaatgcagcatagactactgatgaacagtgcatggcaaagaagccaagtgtcatttcatggcctcagcctctcagctgagaagcagggcacagctcacccaggctaggaaaaacagaggcaagtcctggaaagctgtctgcttttaaccaagagttactggccatcaagtgtcttggttaaaaaataagtgtcaggcaaccttcttggtagatagagtgtgttgggggcgattatcagagtctggtaatgacttctgagggtcccaaagagtgaagtgatatttacatagcaaatccaagganggggattgtgtgcaatatangtggangtgggggcaggttttgtgggtttgccaagctccaaggtcatacaatgtgcatgtcaaggacaagaaatcaaagccatgtgaaatggttggaggtggttcagtttgaggtcatgtgtttctcagctcctgttgtggaattagtgtgagacccagaagactgtggccaaagctattatggacccatggtctctgtggactcatccctcatgccttctgctctctgatcacatccacactcatgtcatcctcgttcttccaaggtgaggttactagcactgcacaggggctgatgagagcatgtcctgccaggaaaaaccatcccaaagagatgctttcccccttggcactgtgtcctgtatttgctcagcagcccacatcctgttctgccccaaaccttggggcagacttcccacaggtgaatttgaactccccaagattaaaatcaagcctgtattcaggaaacacttgggagtcctcgaggttcaccgagagggaagttggaaatttttcacttatgtcagtgcgtttgcagttgggcaacagccagattgttcatatggcaatcaatcaaacacacctaagttttttccacatattagccatcgactgttagcaaaagccctcacttcctttatattgatttatagcagcagcagaaatataccaccctagaggacacacctccttttagctaggtacctataaatgtccaggattttctattcaattgagaagaacccagcaaaatgg

SEQ ID NO: 2 - DNA, 42 bp, Artificial Sequence
  Primer
gaattcacgcgtgatcccagacccagctgcattatcattctc

SEQ ID NO: 3 - DNA, 42 bp, Artificial Sequence
  Primer
gaattcaagcttggtgtgtcctctagggtggtatatttctgc

SEQ ID NO: 4 - DNA, 42 bp, Artificial Sequence
  Primer
gaattcggtaccgcattccactggcctgttttatgttttggg

SEQ ID NO: 5 - DNA, 30 bp, Artificial Sequence
  Primer
gcttctttgccatgcactgttcatcagtag

SEQ ID NO: 6 - DNA, 33 bp, Artificial Sequence
  Primer
caaggtcaagagatcgacaccatcctggccaac

SEQ ID NO: 7 - DNA, 33 bp, Artificial Sequence
  Primer
gttggccaggatggtgtcgatctcttgaccttg

SEQ ID NO: 8 - DNA, 60 bp, Artificial Sequence
  Primer
ggagctagctagaggtcacagtgacctacgagtccctagaggtcacagtgacctacgaga

SEQ ID NO: 9 - DNA, 66 bp, Artificial Sequence
  Primer
tccagatctcgtaggtcactgtgacctctagggactcgtaggtcactgtgacctctagctagctcc

SEQ ID NO: 10 - DNA, 21 bp, Artificial Sequence
  Primer
acggaccatgaccatgaccct

SEQ ID NO: 11 - DNA, 24 bp, Artificial Sequence
  Primer
agctctcagactgtggcagggaaa